(19) United States
(12) Patent Application Publication
(10) Pub. No.: US 2026/0354725 A1
(43) Pub. Date: Nov. 12, 2026

(54) SYSTEM AND METHOD FOR MEASURING BITE FORCE

(71) Applicant: HULTGREN DENTAL TECHNOLOGIES, LLC, Victoria, MN (US)

(72) Inventors: Bruce Willard Hultgren, Victoria, MN (US); Aaron Tucker, Victoria, MN (US)

(21) Appl. No.: 19/669,075

(22) Filed: May 6, 2026

Related U.S. Application Data
(60) Provisional application No. 63/802,334, filed on May 8, 2025.

(51) Int. Cl. A61B 5/22; A61B 5/00; A61C 7/08

(52) U.S. Cl. CPC A61B 5/228; A61B 5/0002; A61B 5/682; A61C 7/08; A61B 2560/0223; A61B 2562/066

(57) ABSTRACT

A device that measures a bite force. The device includes a dental appliance including a sensor array with a polydimethylsiloxane (PDMS) sensor substrate containing at least one electrode. The dental appliance further includes a conductive material that produces a strain gauge effect when a PDMS sensor in the sensor array is subject to a force. The dental appliance further includes a sensor bed layered with at least one layer of PDMS substrate containing the at least one electrode and the conductive material. The device further includes a sensor calibration device with a torque-wrench style press and a 3D printed upper dentition and a 3D printed lower dentition to calibrate to typical forces on the sensor array of the dental appliance. The device further includes a force vector calculation using a strain calculation plus a size of the sensor array.

### CROSS-REFERENCE TO RELATED APPLICATIONS

This application claims the benefit of and priority to U.S. Provisional Patent Application No. 63/802,334, filed May 8, 2025, entitled “BITE FORCE MEASUREMENT DEVICE,” which is incorporated herein by reference in its entirety.

### FIELD OF INVENTION

The present disclosure relates to a system and method for measuring at least bite force, force vectors and bite positions.

### BACKGROUND

The force generated during biting puts pressure on a patient's dentition. In many cases, this bite force is not spread equally from tooth to tooth. Rather, the bite force is concentrated on specific points of contact between a tooth (or teeth) on the upper dentition and a tooth (or teeth) on the lower dentition. Determining bite force between teeth on the upper dentition and teeth on the lower dentition is helpful when determining treatment plans for fabrication of dental restorations, dental malocclusions, bruxism, concussive forces, and other conditions.

As such, there is a need for an accurate device to measure the bite force concentrated on specific points of contact between a tooth (or teeth) on the upper dentition and a tooth (or teeth) on the lower dentition. Such a device could be used in a variety of settings, including but not limited to dental offices during dental restoration treatment planning or as a mouthguard used during contact sports to aid in the diagnosis of concussion.

### SUMMARY

This disclosure is directed to a device for measuring the bite force in concentrated points of contact between a tooth (or teeth) on the upper dentition and a tooth (or teeth) on the lower dentition. In one possible configuration and by non-limiting example, a dental appliance is formed to measure the pressure at various points along the dentition of a patient. In some instances this may be in the form of a standard one-size-fits-all retainer and a 3D printed dentition. While in other instances, this may be in the form of a patient specific retainer.

In one instance, the disclosure includes a device that measures a bite force, the device including a dental appliance, a sensor array with a polydimethylsiloxane (PDMS) sensor substrate containing at least one electrode, a conductive material that produces a strain gauge effect when a PDMS sensor in the sensor array is subject to a force, a sensor bed layered with at least one layer of PDMS substrate containing the at least one electrode and the conductive material, a sensor calibration device with a torque-wrench style press and a 3D printed upper dentition and a 3D printed lower dentition to calibrate to typical forces on the sensor array of the dental appliance, and a force vector calculation using a strain calculation plus a size of the sensor array.

In another instance, the disclosure includes a device that measures a bite force, the device including a sensor array with a polydimethylsiloxane (PDMS) sensor substrate containing at least one electrode, a conductive material that produces a strain gauge effect when a PDMS sensor in the sensor array is subject to a force, a sensor bed layered with at least one layer of PDMS substrate containing the at least one electrode and the conductive material, a wire harness adhered to a custom dentition retainer with at least one PDMS sensor of the sensor array, and a force vector calculation using a strain calculation plus a size of the sensor array.

In yet another instance, the disclosure includes a method of measuring bite force, the method including having a dental appliance, creating a sensor array with a polydimethylsiloxane (PDMS) sensor substrate containing at least one electrode, producing a strain gauge effect when a conductive material with at least one PDMS sensor in the sensor array is subject to a force, layering a sensor bed with at least one layer of PDMS substrate containing the at least one electrode and the conductive material, calibrating to typical forces on the sensor array of the dental appliance using a sensor calibration device with a torque-wrench style press and a 3D printed upper dentition and a 3D printed lower dentition, and calculating a force vector using a strain calculation plus a size of the sensor array.

In yet another instance, the disclosure includes a method of measuring bite force. The method includes having a dental appliance, creating a sensor array with a polydimethylsiloxane (PDMS) sensor substrate containing at least one electrode, producing a strain gauge effect when a conductive material with at least one PDMS sensor in the sensor array is subject to a force, layering a sensor bed with at least one layer of PDMS substrate containing the at least one electrode and the conductive material, adhering a wire harness to a custom dentition retainer with at least one PDMS sensor of the sensor array, and calculating a force vector using a strain calculation plus a size of the sensor array.

In a first aspect, the techniques described herein relate to a device that measures a bite force, the device including: a dental appliance including a bite surface; a sensor array disposed on the bite surface, the sensor array including a size and a flexible elastomeric sensor substrate containing at least one electrode and a conductive material distributed within the elastomeric sensor substrate, wherein the conductive material produces a strain gauge effect when the sensor array is subject to a compressive force; a sensor bed layered with at least one layer of the elastomeric sensor substrate containing the at least one electrode and the conductive material, and at least one non-conductive layer; a sensor calibration device including a torque-wrench style press, a 3D printed upper dentition, and a 3D printed lower dentition configured to apply a calibration force to the sensor array; and a processor configured to calculate a force vector based on a strain calculation derived from the conductive material and the size of the sensor array.

In a second aspect, the techniques described herein relate to a device, wherein the elastomeric sensor substrate includes polydimethylsiloxane (PDMS).

In a third aspect, the techniques described herein relate to a device, wherein the elastomeric sensor substrate includes one or more of: polydimethylsiloxane (PDMS), polyurethane elastomer, styrene-ethylene-butylene-styrene (SEBS), silicone, or a hydrogel.

In a fourth aspect, the techniques described herein relate to a device, wherein the conductive material includes one or more of: silver particles, silver nanowires, gold nanoparticles, carbon nanotubes, graphene, carbon black, copper nanoparticles, a liquid metal alloy, or a conductive polymer.

In a fifth aspect, the techniques described herein relate to a device, wherein the conductive material includes silver particles having a particle size of less than 250 μm.

In a sixth aspect, the techniques described herein relate to a device, wherein the silver particles have a particle size between 2 μm and 3.5 μm, or between 5 μm and 8 μm.

In a seventh aspect, the techniques described herein relate to a device, wherein the at least one electrode receives electrical signals based on a change in electrical resistance in the conductive material caused by a compressive force applied to the sensor array.

In an eighth aspect, the techniques described herein relate to a device, wherein the at least one electrode includes one or more of: gold, platinum, silver, silver-chloride, or carbon.

In a ninth aspect, the techniques described herein relate to a device, wherein the dental appliance further includes an alignment guide configured to align the dental appliance with a patient's dentition.

In a tenth aspect, the techniques described herein relate to a device, further including a wireless communication module configured to transmit electrical signals from the at least one electrode to a remote computing device via one or more of Bluetooth, Wi-Fi, radio frequency identification (RFID), near-field communication (NFC), or radio waves.

In an eleventh aspect, the techniques described herein relate to a device that measures a bite force, the device including: a sensor array including a flexible elastomeric sensor substrate containing at least one electrode and a conductive material distributed within the elastomeric sensor substrate, wherein the conductive material produces a strain gauge effect when the sensor array is subject to a compressive force; a sensor bed layered with at least one layer of the elastomeric sensor substrate containing the at least one electrode and the conductive material, and at least one non-conductive layer; a wire harness mechanically coupled to the sensor bed and configured to be adhered to a patient-specific dental retainer; and a processor configured to calculate a force vector based on a strain calculation derived from the conductive material and a size of the sensor array.

In a twelfth aspect, the techniques described herein relate to a device, wherein the patient-specific dental retainer is a clear custom-made dental retainer.

In an thirteenth aspect, the techniques described herein relate to a device, wherein the patient-specific dental retainer includes a thermoplastic polyurethane (TPU) retainer, a polyethylene terephthalate glycol (PETG) retainer, or a polymethylmethacrylate (PMMA) retainer.

In a fourteenth aspect, the techniques described herein relate to a device, wherein the elastomeric sensor substrate includes polydimethylsiloxane (PDMS).

In a fifteenth aspect, the techniques described herein relate to a device, wherein the conductive material includes one or more of: silver particles, silver nanowires, gold nanoparticles, carbon nanotubes, graphene, carbon black, copper nanoparticles, a liquid metal alloy, or a conductive polymer.

In a sixteenth aspect, the techniques described herein relate to a device, wherein the at least one electrode receives electrical signals based on a change in electrical resistance in the conductive material caused by a compressive force applied by an upper dentition of a patient and a lower dentition of the patient on the sensor array.

In a seventeenth aspect, the techniques described herein relate to a device, further including a wireless communication module replacing the wire harness, the wireless communication module configured to transmit electrical signals from the at least one electrode to a remote computing device via one or more of Bluetooth, Wi-Fi, radio frequency identification (RFID), near-field communication (NFC), or radio waves.

In an eighteenth aspect, the techniques described herein relate to a method of measuring bite force using a calibration dental appliance device, the method including: providing a dental appliance including a bite surface; creating a sensor array including a flexible elastomeric sensor substrate having at least one electrode embedded therein and a conductive material distributed within the elastomeric sensor substrate; layering a sensor bed with at least one layer of the elastomeric sensor substrate and at least one non-conductive layer; disposing the sensor bed on the bite surface of the dental appliance; calibrating the sensor array by applying a calibration force using a sensor calibration device including a torque-wrench style press, a 3D printed upper dentition, and a 3D printed lower dentition; receiving, from the at least one electrode, electrical signals indicative of a change in electrical resistance in the conductive material resulting from a compressive force applied by a patient's bite; and calculating, by a processor, a force vector based on a strain calculation derived from the electrical signals and a size of the sensor array.

In a nineteenth aspect, the techniques described herein relate to a method, wherein the elastomeric sensor substrate includes polydimethylsiloxane (PDMS).

In a twentieth aspect, the techniques described herein relate to a method, wherein the conductive material includes one or more of: silver (Ag) particles, silver nanowires, gold nanoparticles, carbon nanotubes, graphene, carbon black, copper nanoparticles, a liquid metal alloy, or a conductive polymer.

### BRIEF DESCRIPTION OF THE DRAWINGS

FIG. 1 is a top view of a silver impregnated PDMS sensor device according to a disclosed example;

FIG. 2A is a top view of a silver impregnated PDMS sensor device with a sectional view cutaway according to a disclosed example;

FIG. 2B is a sectional view of layered silver impregnated PDMS sensors according to a disclosed example;

FIG. 3 is schematic view of a silver impregnated PDMS sensor device being inserted into a 3D printed dentition within the calibration arbor press according to a disclosed example;

FIG. 4 is a perspective view of another instance of a silver impregnated PDMS sensor device with a silver impregnated PDMS sensor array according to a disclosed example;

FIG. 5 is a schematic view of a silver impregnated PDMS sensor device with a wire harness for mounting to a patient specific retainer according to a disclosed example;

FIG. 6 is a side view of another instance of a silver impregnated PDMS sensor device mounted to a patient specific retainer with the bite force sensor contacts the upper part of the dentition according to a disclosed example;

FIG. 7 is a block diagram of a method of detecting bite force using a silver impregnated PDMS sensor device with a calibration arbor press and a 3D printed dentition according to a disclosed example;

FIG. 8 is a block diagram of a method of detecting bite force using a silver impregnated PDMS sensor device with a custom dentition specific retainer for detecting bite force and/or direction according to a disclosed example;

FIG. 9 is a schematic diagram of an example computing environment that supports the device according to a disclosed example; and

FIG. 10 is a schematic block diagram of a computer system that supports a measuring bite force, according to a disclosed example.

### DETAILED DESCRIPTION

While this disclosure is susceptible of embodiments in many different forms, there are shown in the drawings and is described in detail herein specific embodiments with the understanding that the present disclosure is an exemplification of the principles of the disclosure. It is not intended to limit the disclosure to the specific illustrated embodiments. The features of the disclosure disclosed herein in the description, drawings, and claims can be significant, both individually and in any desired combinations, for the operation of the disclosure in its various embodiments. Features from one instance can be used in other instances.

Before any embodiments are explained in detail, it is to be understood that this disclosure is not limited in its application to the details of construction and the arrangement of components set forth in the following description or illustrated in the attached drawings. The system and method disclosed is capable of other embodiments and of being practiced or of being carried out in various ways. Also, it is to be understood that the phraseology and terminology used herein are for the purpose of description and should not be regarded as limiting. For example, the use of “including,” “comprising,” or “having” and variations thereof herein is meant to encompass the items listed thereafter and equivalents thereof as well as additional items.

As used herein, unless otherwise specified or limited, the terms “mounted,” “connected,” “supported,” and “coupled” and variations thereof are used broadly and encompass both direct and indirect mountings, connections, supports, and couplings. Further, unless otherwise specified or limited, “connected” and “coupled” are not restricted to physical or mechanical connections or couplings.

The present disclosure relates to a dental appliance and method for using the dental appliance for measuring and determining bite force at specific points of contact between a tooth (or teeth) on the upper dentition and a tooth (or teeth) on the lower dentition. The dental appliance may be configured to go between an upper and a lower dentition or adhere to a custom dentition specific retainer. In some instances the dental appliance includes one or more measurement devices or sensors to measure properties relating to the physiology of the patient. For example, in some instances the dental appliance incudes sensors to measure force or pressure on the surface of the dentition. In some instances, information captured by the dental appliance is associated with a dental model of the patient's dentition.

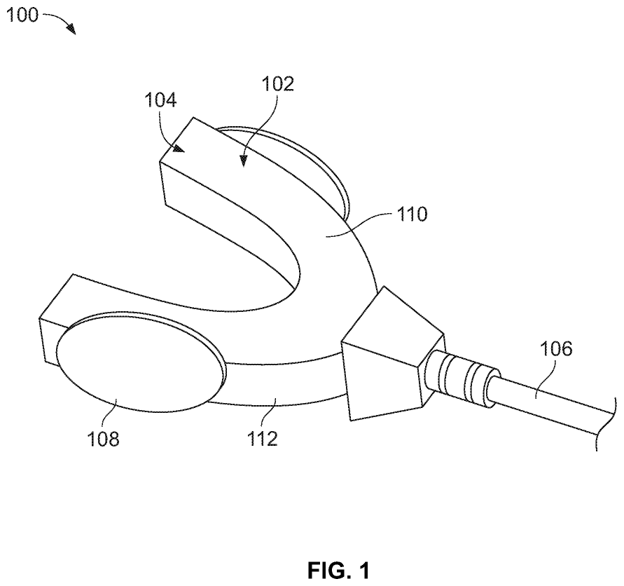

FIG. 1 represents an example of a device 100 in according to the principles of this disclosure. Device 100 may be in the form of a silver impregnated polydimethylsiloxane (PDMS) sensor device. Device 100 may include a dental appliance 102. The dental appliance 102 may include a bite surface 104, a wire harness 106, and an alignment guide 108 of a patient. The bite surface 104 may be designed to be placed between an upper and lower dentition of a patient. The bite surface 104 may further include an upper bite surface portion 110 and a lower bite surface portion 112. The upper bite surface portion 110 may rest against the upper teeth. The lower bite surface portion 112 may rest against the lower teeth. The wire harness 106 may connect a plurality of electrodes to an electronic device for data transfer. The electronic device may include a controller and a processor for processing the received data. In some instances, wire harness 106 may be replaced by a wireless data transfer device that is configured to transfer data via Bluetooth, radio waves, radio frequency identification (RFID), wi-fi, or any other suitable wireless data transfer system. The alignment guide 108 may be configured to align the device 100 between the upper and lower dentitions of a patient such that the alignment guide 108 may assure that the upper dentition rests against the upper bite surface portion 110 and the lower dentition rests against the lower bite surface portion 112. The alignment guide 108 may further ensure that the device 100 is centered between the left and right sides as well as properly centered from the front to the back of the patient's dentition.

In some embodiments, the wire harness 106 is replaced by a wireless communication module integrated into or attached to the dental appliance 102 or patient specific retainer. The wireless communication module may be configured to transmit electrical signals from the electrodes 116 to a remote computing device via Bluetooth (including Bluetooth Low Energy (BLE)), Wi-Fi, radio frequency identification (RFID), near-field communication (NFC), Zigbee, or any other suitable short-range or long-range wireless data transfer protocol. The wireless communication module may be powered by a thin-film rechargeable battery, a supercapacitor, or an energy harvesting element integrated with the device 100. Other power sources may alternatively be used without departing from the principles of this disclosure. Data transmitted wirelessly may be encrypted to protect patient health information in accordance with applicable regulatory requirements. The remote computing device may include, but is not limited to, a smartphone, tablet, laptop, or dedicated clinical workstation running a software application configured to receive, display, and analyze bite force data.

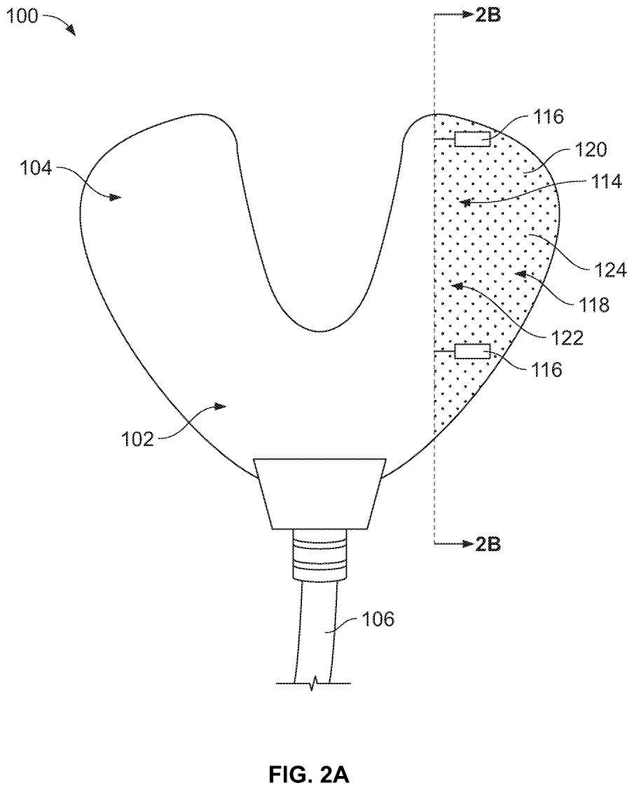

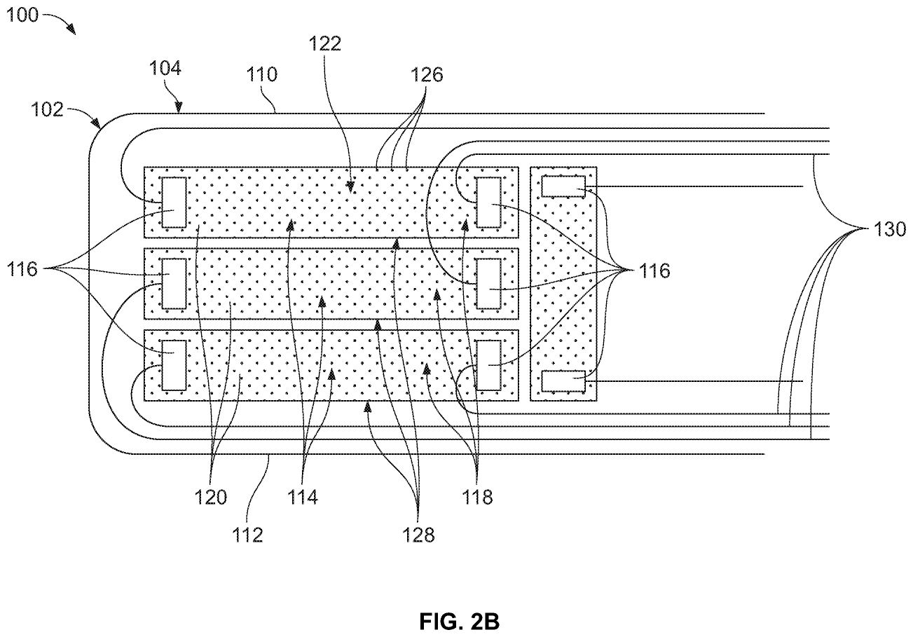

Referring to FIGS. 2A and 2B the device 100 has a sectional view cutaway from dental appliance 102. The dental appliance 102 may further contain a layered sensor bed 114 and a plurality of electrodes 116, among others. The layered sensor bed 114 may comprise a silver (Ag) impregnated PDMS layer 118 and a non-conductive (inert) PDMS layer 120, among others.

Although PDMS is described herein as a preferred non-conductive layer material, it is contemplated that alternative electrically insulating materials may be employed, including but not limited to: Parylene conformal coatings; fluoropolymers such as polytetrafluoroethylene (PTFE) or polyvinylidene fluoride (PVDF); polyimide (PI) film; and thermoplastic elastomers compatible with co-processing alongside the sensor substrate.

In one instance, the silver (Ag) impregnated PDMS layer 118 may include a sensor substrate 122 and a conductive impregnated material 124. In one instance, the sensor substrate 122 may be a liquid PDMS substrate. In one instance, the sensor substrate 122 may be highly flexible and moldable. The sensor substrate 122 may be impregnated with electrically conductive materials. The sensor substrate 122 may have the ability to be cut after formation. The sensor substrate 122 may even be easily adhered to PDMS that is not conductive to form insulating layers or to isolate one sensor from another. The sensor substrate 122 may have electrodes 116 placed during the curing phase. The electrodes 116 receive electrical activity which may be electrical signals and transmit the electrical signals to a controller, which may be located locally or on a remote computing device. The sensor substrate 122 may be reusable, easy to clean and/or maintain.

Although polydimethylsiloxane (PDMS) is described herein as a preferred elastomeric sensor substrate, alternative flexible elastomeric substrate materials may be used without departing from the scope of the disclosure. Such alternatives include, but are not limited to: Ecoflex platinum-catalyzed silicone; polyurethane elastomers; styrene-ethylene-butylene-styrene (SEBS) thermoplastic elastomer; ionic hydrogels such as polyacrylamide-based hydrogels; and silicone. The elastomeric sensor substrate may also be selected to ensure flexibility, biocompatibility under ISO 10993 standards, and resistance to degradation in the oral environment.

The electrodes 116 may be fabricated from any biocompatible electrically conductive material suitable for use in the oral environment. Suitable electrode materials include, but are not limited to: gold (Au); platinum (Pt); silver/silver-chloride (Ag/AgCl); carbon-based electrodes; and copper provided with a biocompatible coating. In some embodiments, non-biocompatible electrically conductive materials may be used without departing from the principles of this disclosure. If non-biocompatible materials used, the conductive material layer may be surrounded by or embedded within a non-conductive layer that is fully biocompatible to avoid leeching the conductive material.

In one instance, the conductive impregnated material 124 may be silver (Ag) particles. In some instances, the silver (Ag) particles 126 may generally be small in size. For example, the silver (AG) particles 126 may be <250 μm. In another instance, for example, the silver (AG) particles 126 may be between 2-3.5 μm or between 5-8 μm. The sensor substrate 122 may be mixed uniformly with silver (Ag) particles 126 forming silver (Ag) impregnated PDMS sensor(s) 128. In some instances, the process of uniformly mixing the silver (Ag) particles 126 with the sensor substrate 122 may include bulk silver (Ag) powder, which may be preliminarily refined by grinding, adding ethanol, and gently stirring the mixture. In some instances, the silver (Ag) particles 126 may be completely dispersed in the liquid mixture by ultrasonication for 30 minutes. In other instances, the silver (Ag) particles 126 may be completely dispersed in the liquid mixture by ultrasonication for a longer or shorter length of time. The ethanol solution on top may be removed with a pipette until the silver (Ag) particles 126 settled at the bottom of the container. When the ethanol is completely volatized, a PDMS prepolymer may be added to the container. In some instances, the PDMS prepolymer may be a Sylgard 160. In another instance, the PDMS prepolymer may be a harder or softer material. The composite may be mixed in a planetary mixer. In some instances, for example, a few drops of a PDMS curing agent may be used between the prepolymer and curing agent. In other instances, more or less PDMS curing agent may be used. The amount and type of PDMS curing agent required may vary according to the manufacturer's instructions. The mixture with the PDMS curing agent may be further mixed again using the planetary mixer. The liquid composite may be poured onto a silicon wafer and spin-coated to form a thin film where the thickness of the film depends on the spin-coating speed and time. In some instances, the thickness may be within a target thickness range of 2-5 mm. In another instance, the thickness may be greater or less than the target thickness. The uncured silver (Ag) impregnated PDMS film may be baked as part of the curing process before being peeled off from the edge of the silicon wafer, thus, forming the silver (Ag) impregnated PDMS sensor(s) 128, which may be easily stretched or twisted and will return to its original state when the tension is fully released.

The uniform mixing of silver (Ag) particles 126 in the sensor substrate 122 may produce a strain gauge effect when PDMS is subjected to forces. In one instance, for example, the conductive impregnated material 124 may have a strain gauge factor of as high as 939 when stretched to 36%. In other instances, the strain gauge factor may be greater or less. In yet other instances, the strain gauge factor may vary based on the location of the device. In one instance, for example, the strain gauge factor may be lower near the molar section of the device to measure greater force in the molar region. The silver (Ag) impregnated PDMS sensor(s) 128 may detect force as low as 0.1 N with a minimum resolution for detection is less than 0.02 N. The strain gauge factor may be the ratio of change in electrical resistance to change in length. The strain gauge factor may be tested and controlled by a high-precision force tester. The strain gauge factor may be used to characterize the sensitivity of the stretchable silver (Ag) impregnated PDMS sensor(s) 128. The results of a resistance change rate and strain with various filling ration and thickness may be characterized in a resistance change rate of

$$\frac{Δ R}{{R}_{0}}$$

versus strain

$$\frac{Δ L}{{L}_{0}} .$$

In general, the resistance change rate increased at each measurement value. In one instance, for example, the resistance change rate and strain with various filling ration may be measured at 150 wt. %, 175 wt. %, and 200 wt. %, while the thickness may be measured at 400 rpm, 600 rpm, and 800 rpm. In this instance, a film having a 175 wt. %-600 rpm may display different sensing characteristics, which may be divided into three different regions. In this instance, for example, Region 1: Gauge Factor (GF) GF₁=10 (ε=0-24%), Region 2: GF₂=71 (ε=24-32%), and Region 3: GF₃=550 (ε=32-36%). In this exemplary instance, Region 1, the resistance may be relatively stable. However, in this exemplary instance, the resistance change rate may drastically increase with each subsequent region. In other instances, the resistance change rate may vary only slightly. In another instance, the film having a 200 wt. %-800 rpm may display different sensing characteristics, which are similarly divided into three different regions. In another instance, for example, Region 1: Gauge Factor (GF) GF₁=9 (ε=0-32%), Region 2: GF₂=50 (ε=32-40%), and Region 3: GF₃=157 (ε=40-48%). In yet other instances, the gauge factor may produce different results depending on the region.

While silver (Ag) particles are described herein as a preferred conductive material, it is expressly contemplated that alternative conductive materials may be used without departing from the scope of the disclosure. Such alternatives include, but are not limited to: silver nanowires (Ag NWs), which form percolation networks at lower fill ratios and may offer improved sensitivity; gold nanoparticles (Au NPs), which provide excellent conductivity, biocompatibility, and corrosion resistance suitable for the oral environment; carbon nanotubes (CNTs), including single-walled (SWCNTs) and multi-walled (MWCNTs) variants, which offer very high conductivity and high gauge factors; graphene and reduced graphene oxide (rGO); carbon black; copper nanoparticles, optionally provided with a biocompatible silver or other passivation coating; gallium-based liquid metal alloys such as EGaIn or Galinstan embedded in the elastomeric substrate to enable extremely high stretchability without fracture of conductive pathways; and conductive polymers such as PEDOT:PSS. The selected conductive material should be biocompatible, should not leach harmful ions in the oral environment, and should produce a measurable strain gauge effect when the sensor substrate is subjected to compressive force.

The silver (Ag) impregnated PDMS layer 118 may produce a field of strains which are converted to forces by a controller on a computing device using a software. In an instance, the data processing unit may be configured to convert the strain to stress using modulus of elasticity of the PDMS. The modulus may be a design factor similar to the hardness of the material. The data processing unit may be configured to convert stress to force by multiplying by the surface area of the sensor. The plurality of silver (Ag) impregnated PDMS sensor(s) 128 may be arranged in an array. The array may be in a variety of orientations to capture various bite forces and shear forces. The arrangement of the plurality silver (Ag) impregnated PDMS sensor(s) 128 in an array in FIGS. 2A and 2B is merely exemplary and is not meant to be limiting.

Further in an instance, the electrodes 116 may be arranged in an array in a variety of orientations to capture electrical voltage changes to quantify the strain on the silver (Ag) impregnated PDMS sensor(s) 128. The electrodes 116 may be connected via wire harness leads 130. The wire harness leads 130 may be further connected to the wire harness 106. The electrodes 116 may receive a signal(s) from the silver (Ag) impregnated PDMS sensor(s) 128 and transmit the signal(s) via the wire harness leads 130 to the wire harness 106. The wire harness 106 may be connected to a controller with a processor for processing the signal(s). The controller may automatically receive the signal(s) via the wire harness 106. The processor of the controller may automatically process the received signal(s), which represent a change in electrical voltage and processes the change in electrical voltage in order to calculate the strain and produce the strain calculation value. In one instance, the processor may estimate the force in the z direction when a patient bites down on each individual silver (Ag) impregnated PDMS sensor(s) 128 in the array using the strain calculation value and then adding the size of the silver (Ag) impregnated PDMS sensor(s) 128. In another instance, the processor may estimate the force and/or vector of travel of any particular point on the tooth as it moves functionally against the opposing tooths surface. For example, in in the x, y, directions to measure the trajectory when a patient bites down on each individual silver (Ag) impregnated PDMS sensor(s) 128 in the array using the strain calculation value and add the size of the silver (Ag) impregnated PDMS sensor(s) 128. The trajectory of the angle of the teeth coming together, which is referred to as the vector of travel, may allow for the calculation of a more accurate measurement of the screw axis of the jaw. The screw axis, for example, may be on the axis of a four-bar-linkage design or linkage design. The strain calculation value may be added to the size of the silver (Ag) impregnated PDMS sensor(s) 128 to generate an output value. The output value may be transmitted from the controller to a display for a user. The display may be, but is not limited to, an interactive screen, a touchscreen, a graphical user interface, or the like. The user may interpret the output value and/or vector of travel of any particular point on the tooth as it moves functionally against the opposing tooths surface to determine the force of a patient's bite. The output value and/or vector of travel is interpreted by a user at various pinpointed locations of the patient's dentition based on the bite force calculated for each silver (Ag) impregnated PDMS sensor(s). In one instance, for example, a user may analyze changes in bite force over time at specific points of the patient's dentition. In another instance, a comprehensive bite analysis may be produced to evaluate and analyze the patient's dentition for various issues. The force calculated for each silver (Ag) impregnated PDMS sensor(s) may provide a more accurate determination, pinpointed at various points of the patient's dentition. In some embodiments, the output value may be used to assist a clinician in the selection of appropriate biomaterials to be used in dental restorations. Further, the output value may be used to assess restoration outcomes, including, but not limited to, balance of bite, avoiding occlusion of injured teeth-balanced bite or injured teeth that need to be out of occlusion when no contact or very light contact is desired.

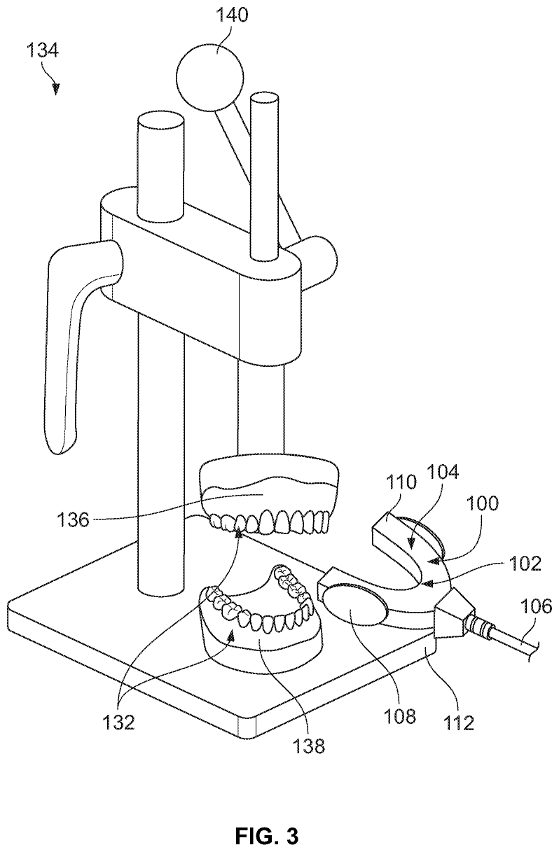

Referring to FIG. 3, a dental appliance 102 may be inserted into a 3D printed dentition 132 within the calibration arbor press 134. The dental appliance 102 may be an example of, or may include aspects of, dental appliance 102 of FIGS. 1-3. The dental appliance 102 may include a bite surface 104, a wire harness 106, and an alignment guide 108. The 3D printed dentition 132 may be created by taking a mold of a patient's teeth and generating a 3D model from the mold of the patient's teeth. The 3D printed dentition 132 may include an upper dentition portion 136 and a lower dentition portion 138.

The 3D printed upper dentition and lower dentition are preferably fabricated from a rigid or semi-rigid dental-grade resin, such as a stereolithography (SLA) biocompatible resin or polymethylmethacrylate (PMMA), selected to have a hardness sufficient to transmit calibration forces to the sensor array without deformation, while remaining biocompatible in the event of patient contact.

The bite surface 104 may include an upper bite surface portion 110 that makes contact with the upper dentition portion 136 and a lower bite surface portion 112 that makes contact with the lower dentition portion 138 of the 3D printed dentition 132. Once the 3D printed dentition 132 may be placed on the calibration arbor press 134 and the dental appliance 102 may be inserted such that contact is made between the bite surface 104 and the upper dentition portion 136 and lower dentition portion 138 of the 3D printed dentition 132, the silver (Ag) impregnated PDMS sensor(s) 128 may be calibrated to typical forces.

The calibration arbor press 134 may contain a torque limited calibration handle 140 which operates as a torque-wrench style press. The torque limited calibration handle 140 may be lowered pressing the upper dentition portion 136 and lower dentition portion 138 of the 3D printed dentition 132 onto the upper bite surface portion 110 and lower bite surface portion 112, respectively, of the dental appliance 102. By pressing the upper dentition portion 136 and lower dentition portion 138 of the 3D printed dentition 132 onto the upper bite surface portion 110 and lower bite surface portion 112 of the dental appliance 102, the 3D printed dentition 132 may produce a typical scenario for the PDMS sensor array to detect the bite force between the upper dentition portion 136 and lower dentition portion 138 of the 3D printed dentition 132.

The calibration arbor press 134 may be fabricated from stainless steel, aluminum alloy, or another rigid, corrosion-resistant material. The torque limited calibration handle 140 is preferably configured to apply a controlled torque within the range of 0.5 to 10 Newton-meters (N·m), selectable based on the calibration force required for a given sensor configuration, thereby ensuring reproducible and standardized calibration forces across sensor array batches.

The silver (Ag) impregnated PDMS sensor(s) 128 may detect the bite force at specific points of the upper dentition portion 136 and lower dentition portion 138 of the 3D printed dentition 132 by detecting the force on the sensors on various points of the silver (Ag) impregnated PDMS sensor(s) 128 positioned in an array. In some instances, the silver (Ag) impregnated PDMS sensor(s) 128 may detect the bite force in the z axis direction to detect the force that the teeth of the upper dentition portion 136 have on the lower dentition portion 138 are when they come together during a bite. In other instances, the silver (Ag) impregnated PDMS sensor(s) 128 may detect the bite force in the x and y axes directions to detect the trajectory of the angle the teeth of the upper dentition portion 136 and the lower dentition portion 138 are coming together during a bite. Each of the silver (Ag) impregnated PDMS sensor(s) 128 may produce a change in electrical voltage which is used to quantify the strain on the upper dentition portion 136 and lower dentition portion 138. The change in electrical voltage may be transmitted via the electrodes 116 through the wire harness 106 to a computing device. The computing device may include a controller, which further contains a processor. The controller may receive the change in electrical voltage from the electrodes 116 via the wire harness 106. The processor may use the change in electrical voltage to calculate the strain and produce the strain calculation value. The force on each individual silver (Ag) impregnated PDMS sensor(s) 128 in the array may be estimated from the strain calculation value and then added to the size of the silver (Ag) impregnated PDMS sensor(s) 128.

The strain calculation value may be added to the size of the silver (Ag) impregnated PDMS sensor(s) 128 to generate the output value. The output value may be transmitted from the controller to a display for a user. The display may be, but is not limited to, an interactive screen, a touchscreen, a graphical user interface, or the like. The user may interpret the output value and/or vector of travel of any particular point on the tooth as it moves functionally against the opposing tooths surface to determine the force of a patient's bite. The output value is interpreted by a user at various pinpointed locations of the patient's dentition based on the bite force calculated for each silver (Ag) impregnated PDMS sensor(s). In one instance, for example, a user may analyze changes in bite force and/or direction over time at specific points of the patient's dentition. In another instance, the output value may be used to evaluate and assess the bite force over a period of time. In an instance, for example, a comprehensive bite analysis may be produced to evaluate and analyze the patient's dentition for various issues. In one instance, for example, it may be determined from the comprehensive bite analysis—which may show a spike (hitch, jump, motion perturbation, etc.) in the curvilinear motion of the temporomandibular joint (the jaw joint)—that the joint is not functioning properly. In some embodiments, the comprehensive bite analysis can identify non-harmonious-unequal-forces present amongst adjacent teeth, which may alert the dentist to harmonize those discrepancies with therapeutic intervention, e.g. restorations, orthodontics, splints, etc. In another instance, for example, the comprehensive bite analysis may indicate another underlying disease. In one instance, the force calculated for each silver (Ag) impregnated PDMS sensor(s) may provide a more accurate determination of the direct force on each tooth, which may be measured in the z-axis direction, pinpointed at various points of the patient's dentition. In another instance, the combination of forces calculated temporally for each silver (Ag) impregnated PDMS sensor(s) may provide for an accurate determination of the trajectory of the angle of the teeth coming together during a bite or vector of travel of any particular point on the tooth as it moves functionally against the opposing tooths surface. The forces may be measured in the x or y-axis direction(s), combined with the measurements of force in the z-axis direction, at various points of the patient's dentition. In certain instances, measuring the trajectory of the teeth coming together as a patient bites down or vector of travel of any particular point on the tooth as it moves functionally against the opposing tooths surface may allow for the calculation of the screw axis of the jaw. In some embodiments, the screw axis may be on the axis of a four-bar-linkage design or linkage design. In some instances, the force vectors may measure the protrusive and excursive forces present as the patient bites down. Further analysis of the force vectors may allow for a determination of a centric relation of the condyle of the mandible to the temporomandibular joint.

In certain embodiments, the computing device executing bite force analysis may incorporate machine learning or artificial intelligence (AI) algorithms to identify patterns in bite force data. For example, a trained machine learning model may be used to classify bite force patterns associated with known temporomandibular joint (TMJ) disorders, bruxism, concussion, or other dental or neurological conditions. Training data may be derived from a population of patients with known diagnoses. The machine learning model may comprise a neural network, support vector machine, random forest classifier, or other suitable algorithm. In such embodiments, the system may generate a diagnostic suggestion or alert for clinician review based on the patient's measured bite force profile.

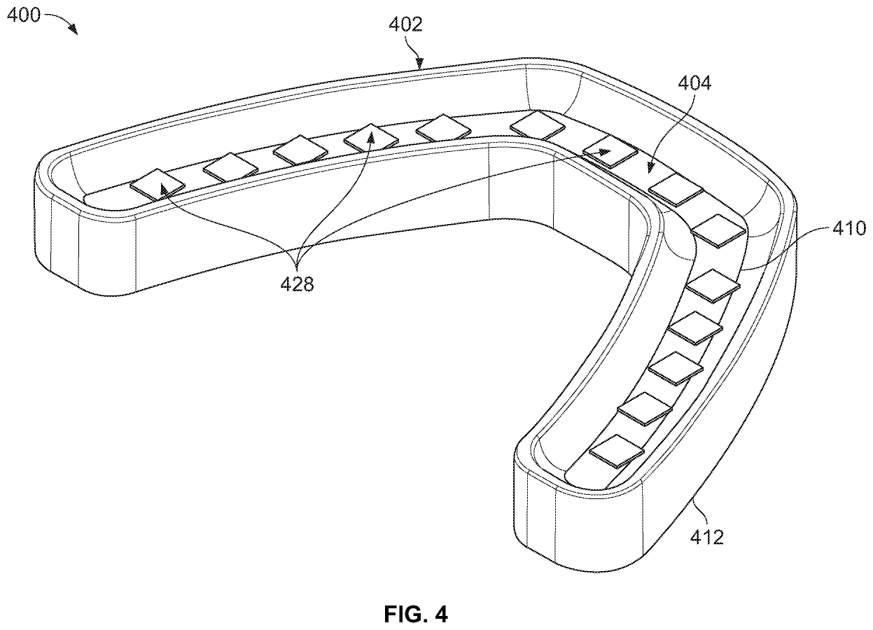

FIG. 4 represents an example of a bite force measurement device as described in FIG. 1 through FIG. 3. FIG. 4 is a perspective view of a device 400 containing an inert dental appliance 402 with a plurality of silver (Ag) impregnated PDMS sensor(s) 428 arranged in an array. The dental appliance 402 further may include a bite surface 404 with an upper bite surface portion 410 and a lower bite surface portion 412. Dental appliance 402 may have any desired shape, including a bite surface 404 having a patient specific contour, without departing from the principles of this disclosure. Such patient specific contour may be desirable if the dental appliance 402 is used as a mouthguard for contact sports, in one non-limiting example. In one embodiment, the morphology of the dental appliance can be adapted to the desired hardness of the material used to produce dental appliance 402.

The plurality of silver (Ag) impregnated PDMS sensor(s) 428 may be arranged in an array along the bite surface 404. In this instance, the plurality of silver (Ag) impregnated PDMS sensor(s) 428 may be arranged in an array along the bite surface 404 of dental appliance 402, thereby having silver (Ag) impregnated PDMS sensor(s) 428 near the front of the bite surface 404 where the incisors are positioned in the mouth, further including silver (Ag) impregnated PDMS sensor(s) 428 near the left and right sides of the bite surface 404 where the bicuspid or the pre-molars are positioned in the mouth, and in the left and right back portions of the bite surface 404 where the molars are positioned in the mouth. The array arrangement of FIG. 4 is merely exemplary and is not meant to be limiting.

The silver (Ag) impregnated PDMS sensor(s) 428 may detect the bite force at specific points on the upper bite surface portion 410 and lower bite surface portion 412 of the device 400 by detecting the force on the sensors on various points of the silver (Ag) impregnated PDMS sensor(s) 428 positioned in an array. In some instances, the silver (Ag) impregnated PDMS sensor(s) 428 may detect the bite force in the z axis direction to detect the force that the teeth will have on the upper bite surface portion 410 and on the lower bite surface portion 412 when the teeth come together during a bite. In other instances, the silver (Ag) impregnated PDMS sensor(s) 428 may detect the bite force in the x and y axes directions to detect the trajectory of the angle the teeth come together during a bite and/or vector of travel of any particular point on the tooth as it moves functionally against the opposing tooths surface, such as between the upper bite surface portion 410 and the lower bite surface portion 412. The silver (Ag) impregnated PDMS sensor(s) 428 may detect the force of a patient's bite at various pinpointed locations of the patient's dentition based on the bite force calculated for each silver (Ag) impregnated PDMS sensor(s) 428. In one instance, the force calculated for each silver (Ag) impregnated PDMS sensor(s) 428 may provide a more accurate determination of the direct force on each tooth and/or vector of travel of any particular point on the tooth as it moves functionally against the opposing tooths surface. The vector of travel, for example, may be measured in the z-axis direction, pinpointed at various points of the patient's dentition. In another instance, the combination of forces calculated temporally for each silver (Ag) impregnated PDMS sensor(s) 428 may provide for an accurate determination of the trajectory of the angle of the teeth coming together during a bite and/or vector of travel of any particular point on the tooth as it moves functionally against the opposing tooths surface. The forces, may be measured in the x or y-axis direction(s), combined with the measurements of force in the z-axis direction, at various points of the patient's dentition. In certain instances, measuring the trajectory of the teeth coming together as a patient bites down or vector of travel of any particular point on the tooth as it moves functionally against the opposing tooths surface may allow for the calculation of the screw axis of the jaw. The screw axis, for example, may be on the axis of a four-bar-linkage design or linkage design.

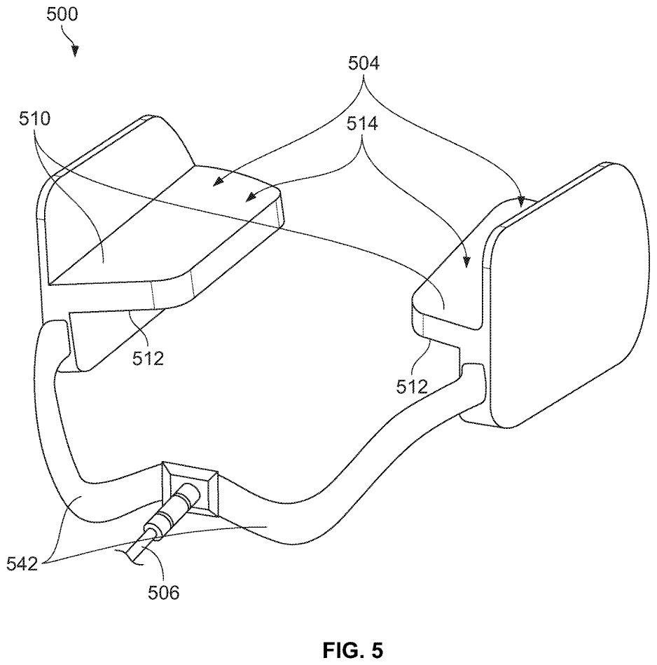

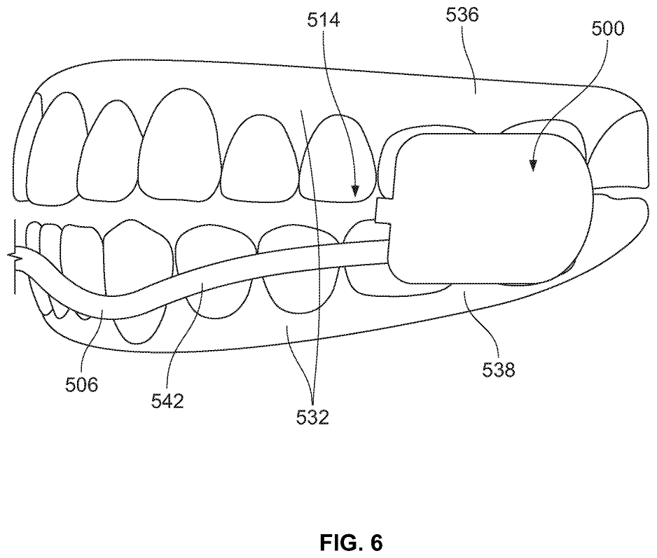

In yet another instance, FIGS. 5 and 6 illustrate a device 500 that uses silver (Ag) impregnated PDMS sensor(s) to determine force exerted by a patient's bite based on an estimate the force on the silver (Ag) impregnated PDMS sensor(s). FIG. 5 illustrates a top view of device 500. FIG. 6 illustrates a side view of device 500 mounted to a patient specific retainer 532. The device 500 includes a bite surface 504, a wire harness 506, a sensor bed 514, a wire harness adherence surface 542.

The patient specific retainer 532 may be formed by taking a mold of a patient's teeth and generating a patient specific retainer 532 from the mold of the patient's teeth. The patient specific retainer 532 may include an upper portion of the patient specific retainer 536 and a lower portion of the patient specific retainer 538. The bite surface 504 may include an upper bite surface portion 510, and a lower bite surface portion 512.

The upper bite surface portion 510 may make contact with the upper portion of the patient specific retainer 536 and the lower bite surface portion 512 that makes contact with the lower portion of a patient specific retainer 538. When the device 500 is placed between the upper portion of the patient specific retainer 536 and the lower portion of the patient specific retainer 538 such that contact may be made between the upper portion of the patient specific retainer 536 and the upper bite surface portion 510, and between the lower portion of the patient specific retainer 538 and the lower bite surface portion 512, the silver (Ag) impregnated PDMS sensor(s) may be calibrated to typical forces. In some instances, the silver (Ag) impregnated PDMS sensor(s) 528 may detect the bite force in the z axis direction to detect the force that the teeth of the upper dentition portion 536 have on the lower dentition portion 538 are when they come together during a bite. In other instances, the silver (Ag) impregnated PDMS sensor(s) 528 may detect the bite force in the x and y axes directions to detect the trajectory of the angle the teeth of the upper dentition portion 536 and the lower dentition portion 138 are coming together during a bite. In certain instances, measuring the trajectory of the teeth coming together as a patient bites down may allow for the calculation of the screw axis of the jaw. The screw axis, for example, may be on the axis of a four-bar-linkage design or linkage design.

Further in another instance, the wire harness 506 may be detachable. The detachable wire harness 506 may be removed from the sensor bed 514. The detachable wire harness may be, but is not limited to, a rigid wire harness. The detachable wire harness 506 is communicatively connected to a controller for data transfer. The wire harness 506 may transmit the data to a controller or other computing device for processing. The wire harness 506 may connect a plurality of electrodes of the sensor device 500 to an electronic device for data transfer. The electronic device may include a controller and a processor for processing the data.

Further in another instance, the sensor bed 514 may include a silver (Ag) impregnated PDMS layer and a non-conductive layer. The silver (Ag) impregnated PDMS layer may include a sensor substrate and a conductive impregnated material. In an instance, the sensor substrate may be a liquid PDMS substrate. In an instance, the sensor substrate may be highly flexible and moldable. The sensor substrate may be impregnated with electrically conductive materials. The sensor substrate may have the ability to be cut after formation. The sensor substrate may even be easily adhered to itself. The sensor substrate may have electrodes placed during the curing phase. The electrodes receive electrical activity which may be electrical signals and transmit the electrical signals to a controller, which may be located locally or on a remote computing device. The sensor substrate may be reusable, easy to clean and/or maintain clean.

Further in an instance, the conductive impregnated material may include small silver (Ag) particles. The sensor substrate may be mixed uniformly with silver (Ag) particles forming silver (Ag) impregnated PDMS sensor(s). In some instances, the silver (AG) particles 126 may be <250 μm. In another instance, for example, the silver (AG) particles 126 may be between 2-3.5 μm or between 5-8 μm. The process of uniformly mixing the silver (Ag) particles with the sensor substrate may include bulk silver (Ag) powder, which may be preliminarily refined by grinding, adding ethanol, and gently stirring the mixture. In some instances, the silver (Ag) particles may be completely dispersed in the liquid mixture by ultrasonication for 30 minutes. In other instances, the silver (Ag) particles 126 may be completely dispersed in the liquid mixture by ultrasonication for a longer or shorter length of time. The ethanol solution on top may be removed with a pipette until the silver (Ag) particles settled at the bottom of the container. When the ethanol is completely volatized, a PDMS prepolymer may be added to the container. In some instances, the PDMS prepolymer may be a Sylgard 160. In another instance, the PDMS prepolymer may be a harder or softer material. In some instances, the composite may be mixed in a planetary mixer. In some instances, for example, a few drops of a PDMS curing agent may be used between the prepolymer and curing agent. In other instances, more or less PDMS curing agent may be used. The amount and type of PDMS curing agent required may vary according to the manufacturer's instructions. The mixture with the PDMS curing agent may be further mixed again using the planetary mixer. The liquid composite may be poured onto a silicon wafer and spin-coated to form a thin film where the thickness of the film depends on the spin-coating speed and time. In some instances, the thickness may be within a target thickness range of 2-5 mm. In another instance, the thickness may be greater or less than the target thickness. The uncured silver (Ag) impregnated PDMS film may be baked as part of the curing process before being peeled off from the edge of the silicon wafer, thus, forming the silver (Ag) impregnated PDMS sensor(s), which may be easily stretched or twisted and will return to its original state when the tension is fully released.

The uniform mixing of silver (Ag) particles in the sensor substrate may produce a strain gauge effect when PDMS is subjected to forces. In one instance, for example, the conductive impregnated material may have a strain gauge factor of as high as 939 when stretched to 36%. In other instances, the strain gauge factor may be greater or less. In yet other instances, the strain gauge factor may vary based on the location of the device. In one instance, for example, the strain gauge factor may be lower near the molar section of the device to measure greater force in the molar region. The silver (Ag) impregnated PDMS sensor(s) may detect force as low as 0.1 N with a minimum resolution for detection may be less than 0.02 N. The strain gauge factor may be the ratio of change in electrical resistance to change in length. The strain gauge factor may be tested and controlled by a high-precision force tester. The strain gauge factor may be used to characterize the sensitivity of the stretchable silver (Ag) impregnated PDMS sensor(s). The results of a resistance change rate and strain with various filling ration and thickness may be characterized in a resistance change rate of

$$\frac{Δ R}{{R}_{0}}$$

versus strain

$$\frac{Δ L}{{L}_{0}} .$$

In general, the resistance change rate may increase at each measurement value. In one instance, for example, the resistance change rate and strain with various filling ration may be measured at 150 wt. %, 175 wt. %, and 200 wt. %, while the thickness may be measured at 400 rpm, 600 rpm, and 800 rpm. In this instance with a film having a 175 wt. %-600 rpm may display different sensing characteristics, which may be divided into three different regions. In this instance, for example, Region 1: Gauge Factor (GF) GF₁=10 (ε=0-24%), Region 2: GF₂=71 (ε=24-32%), and Region 3: GF₃=550 (ε=32-36%). In this exemplary instance, Region 1, the resistance is relatively stable. However, in this exemplary instance, the resistance change rate may drastically increase with each subsequent region. In other instances, the resistance change rate may vary only slightly. In another instance, the film having a 200 wt. %-800 rpm may display different sensing characteristics, which are similarly divided into three different regions. In another instance, for example, Region 1: Gauge Factor (GF) GF₁=9 (ε=0-32%), Region 2: GF₂=50 (ε=32-40%), and Region 3: GF₃=157 (ε=40-48%). In yet other instances, the gauge factor may produce different results depending on the region.

The silver (Ag) impregnated PDMS layer may produce a field of strains which are converted to forces by a controller on a computing device using a software. In an instance, the data processing unit may be configured to convert the strain to stress using modulus of elasticity of the PDMS. The modulus may be a design factor similar to the hardness of the material. The data processing unit may be configured to convert stress to force by multiplying by the surface area of the sensor. The plurality of silver (Ag) impregnated PDMS sensor(s) may be arranged in an array. The array may be in a variety of orientations to capture various bite forces and shear forces.

Further in another instance, the electrodes may be arranged in an array in a variety of orientations to capture electrical voltage changes to quantify the strain on the silver (Ag) impregnated PDMS sensor(s). The electrodes may be connected via wire harness leads. The wire harness leads may further be connected to the wire harness 506. The electrodes may receive a signal(s) from the silver (Ag) impregnated PDMS sensor(s) and transmit the signal(s) via the wire harness leads to the wire harness 506. The wire harness 506 may be connected to a controller with a processor for processing the signal(s). The controller automatically receives the signal(s) via the wire harness 506. The processor of the controller automatically may process the received signal(s), which represent a change in electrical voltage and processes the change in electrical voltage in order to calculate the strain and produce the strain calculation value. The processor may estimate the force on each individual silver (Ag) impregnated PDMS sensor(s) in the array using the strain calculation value and then adding the size of the silver (Ag) impregnated PDMS sensor(s). The strain calculation value may be added to the size of the silver (Ag) impregnated PDMS sensor(s) to generate an output value. The output value may be transmitted from the controller to a display for a user. The display may be, but is not limited to, an interactive screen, a touchscreen, a graphical user interface, or the like. The user may interpret the output value to determine the force and/or direction of a patient's bite at various pinpointed locations of the patient's dentition based on the bite force calculated for each silver (Ag) impregnated PDMS sensor(s). The force calculated for each silver (Ag) impregnated PDMS sensor(s) provides a more accurate determination, pinpointed at various points of the patient's dentition.

The wire harness adherence surface 542 may be adhered to a patient specific retainer 532. In an instance, the patient specific retainer may be, but is not limited to, a clear custom-made retainer, such as Invisalign.

The dental appliance 102 and/or patient specific retainer 532 may be fabricated from any biocompatible material suitable for use as a dental appliance, including but not limited to: thermoplastic polyurethane (TPU), which is flexible, biocompatible, and suitable for 3D printing; polyethylene terephthalate glycol (PETG); acrylonitrile butadiene styrene (ABS); polymethylmethacrylate (PMMA, acrylic); biocompatible UV-curable dental-grade stereolithography (SLA) resins; and flexible silicone. The selected material should be biocompatible in accordance with ISO 10993, resistant to salivary degradation, and dimensionally stable under occlusal loading conditions.

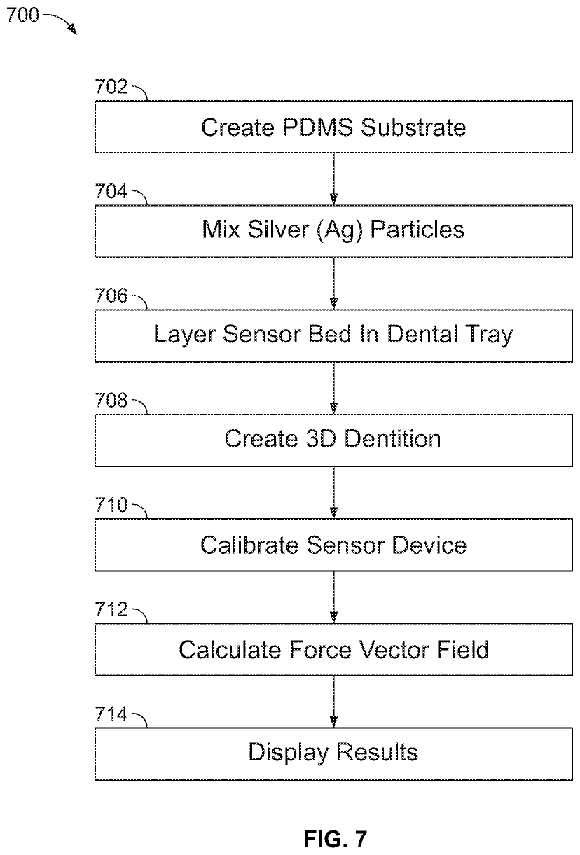

FIG. 7 illustrates a block diagram of a method 700 of detecting bite force using a sensor substrate with a conductive impregnated material in a sensor bed of a dental appliance with a 3D printed dentition and a calibration arbor press.

In method 700, at step 702, the sensor substrate may be created. In one instance, the sensor substrate may be a liquid PDMS substrate. In one instance, the sensor substrate may be highly flexible and moldable. The sensor substrate may be impregnated with electrically conductive materials. The sensor substrate may have the ability to be cut after formation. The sensor substrate may even be easily adhered to itself. The sensor substrate may have electrodes placed during the curing phase. The electrodes receive electrical activity which may be electrical signals and transmit the electrical signals to a controller, which may be located locally or on a remote computing device. The sensor substrate may be reusable, easy to clean and/or maintain clean.

At step 704, the sensor substrate may be mixed with a conductive impregnated material forming a silver (Ag) impregnated PDMS layer. The conductive impregnated material may be silver (Ag) particles. In some instances, the silver (Ag) particles may generally be small in size. For example, the silver (AG) particles 126 may be <250 μm. In another instance, for example, the silver (AG) particles 126 may be between 2-3.5 μm or between 5-8 μm. The sensor substrate may be mixed uniformly with silver (Ag) particles forming silver (Ag) impregnated PDMS sensor(s). In some instances, the process of uniformly mixing the silver (Ag) particles with the sensor substrate may include bulk silver (Ag) powder, which may be preliminarily refined by grinding, adding ethanol, and gently stirring the mixture. In some instances, the silver (Ag) particles may be completely dispersed in the liquid mixture by ultrasonication for 30 minutes. In other instances, the silver (Ag) particles 126 may be completely dispersed in the liquid mixture by ultrasonication for a longer or shorter length of time. The ethanol solution on top may be removed with a pipette until the silver (Ag) particles settled at the bottom of the container. When the ethanol is completely volatized, a PDMS prepolymer may be added to the container. In some instances, the PDMS prepolymer may be a Sylgard 160. In another instance, the PDMS prepolymer may be a harder or softer material. The composite may be mixed in a planetary mixer. In some instances, for example, a few drops of a PDMS curing agent may be used between the prepolymer and curing agent. In other instances, more or less PDMS curing agent may be used. The amount and type of PDMS curing agent required may vary according to the manufacturer's instructions. The mixture with the PDMS curing agent may be further mixed again using the planetary mixer for. The liquid composite may be poured onto a silicon wafer and spin-coated to form a thin film where the thickness of the film depends on the spin-coating speed and time. In some instances, the thickness may be within a target thickness range of 2-5 mm. In another instance, the thickness may be greater or less than the target thickness. The uncured silver (Ag) impregnated PDMS film may be baked as part of the curing process before being peeled off from the edge of the silicon wafer, thus, forming the silver (Ag) impregnated PDMS sensor(s), which may be easily stretched or twisted and will return to its original state when the tension is fully released.

The uniform mixing of silver (Ag) particles in the sensor substrate may produce a strain gauge effect when PDMS is subjected to forces. In one instance, for example, the conductive impregnated material may have a strain gauge factor of as high as 939 when stretched to 36%. In other instances, the strain gauge factor may be greater or less. In yet other instances, the strain gauge factor may vary based on the location of the device. In one instance, for example, the strain gauge factor may be lower near the molar section of the device to measure greater force in the molar region. The silver (Ag) impregnated PDMS sensor(s) may detect force as low as 0.1 N with a minimum resolution for detection may be less than 0.02 N. The strain gauge factor may be the ratio of change in electrical resistance to change in length. The strain gauge factor may be tested and controlled by a high-precision force tester. The strain gauge factor may be used to characterize the sensitivity of the stretchable silver (Ag) impregnated PDMS sensor(s). The results of a resistance change rate and strain with various filling ration and thickness may be characterized in a resistance change rate of

$$\frac{Δ R}{{R}_{0}}$$

versus strain

$$\frac{Δ L}{{L}_{0}} .$$

In one instance, for example, the resistance change rate and strain with various filling ration may be measured at 150 wt. %, 175 wt. %, and 200 wt. %, while the thickness may be measured at 400 rpm, 600 rpm, and 800 rpm. In general, the resistance change rate increased at each measurement value. In this instance with a film having a 175 wt. %-600 rpm may display different sensing characteristics, which may be divided into three different regions. In this instance, for example, Region 1: Gauge Factor (GF) GF₁=10 (ε=0-24%), Region 2: GF₂=71 (ε=24-32%), and Region 3: GF₃=550 (ε=32-36%). In this exemplary instance, Region 1, the resistance may be relatively stable. However in this instance, the resistance change rate may drastically increase with each subsequent region. In other instances, the resistance change rate may vary only slightly. In another instance, the film having a 200 wt. %-800 rpm may display different sensing characteristics, which are similarly divided into three different regions. In another instance, for example, Region 1: Gauge Factor (GF) GF₁=9 (ε=0-32%), Region 2: GF₂=50 (ε=32-40%), and Region 3: GF₃=157 (ε=40-48%). In yet other instances, the gauge factor may produce different results depending on the region.

The silver (Ag) impregnated PDMS layer may produce a field of strains which are converted to forces by a controller on a computing device using a software. In an instance, the data processing unit may be configured to convert the strain to stress using modulus of elasticity of the PDMS. The modulus may be a design factor similar to the hardness of the material. The data processing unit may be configured to convert stress to force by multiplying by the surface area of the sensor. The plurality of silver (Ag) impregnated PDMS sensor(s) may be arranged in an array. The array may be in a variety of orientations to capture various bite forces and shear forces.

Further in another instance, the electrodes may be arranged in an array in a variety of orientations to capture electrical voltage changes to quantify the strain on the silver (Ag) impregnated PDMS sensor(s). The electrodes may be connected via wire harness leads to the wire harness. The electrodes may receive a signal(s) from the silver (Ag) impregnated PDMS sensor(s) and transmit the signal(s) via the wire harness leads to the wire harness. The wire harness is connected to a controller with a processor for processing the signal(s). The controller automatically receives the signal(s) via the wire harness. The processor of the controller automatically processes the received signal(s), which represent a change in electrical voltage and processes the change in electrical voltage in order to calculate the strain and produce the strain calculation value. The processor may estimate the force and/or vector of travel of any particular point on the tooth as it moves functionally against the opposing tooths surface. For example, in each individual silver (Ag) impregnated PDMS sensor(s) in the array using the strain calculation value and then adding the size of the silver (Ag) impregnated PDMS sensor(s). The strain calculation value may be added to the size of the silver (Ag) impregnated PDMS sensor(s) to generate an output value. The output value may be transmitted from the controller to a display for a user. The display may be, but is not limited to, an interactive screen, a touchscreen, a graphical user interface, or the like. The user may interpret the output value and/or vector of travel of any particular point on the tooth as it moves functionally against the opposing tooths surface to determine the force of a patient's bite. The output value and/or vector of travel is interpreted by a user at various pinpointed locations of the patient's dentition based on the bite force calculated for each silver (Ag) impregnated PDMS sensor(s). The output value may be used to assess the health or function of the dentition. In one instance, for example, a user may analyze changes in bite force over time at specific points of the patient's dentition. In another instance, a comprehensive bite analysis may be produced to evaluate and analyze the patient's dentition for various issues. The force calculated for each silver (Ag) impregnated PDMS sensor(s) may provide a more accurate determination, pinpointed at various points of the patient's dentition.

At step 706, the sensor bed of a dental appliance may be created by layering the silver (Ag) impregnated PDMS sensor(s) with a non-conductive (inert) PDMS layers. The silver (Ag) impregnated PDMS layer may produce a field of strains which are converted to forces by a controller on a computing device using a software. In an instance, the data processing unit may be configured to convert the strain to stress using modulus of elasticity of the PDMS. The modulus may be a design factor similar to the hardness of the material. The data processing unit may be configured to convert stress to force by multiplying by the surface area of the sensor. The plurality of silver (Ag) impregnated PDMS sensor(s) may be arranged in an array along the bite surface of the dental appliance, thereby having silver (Ag) impregnated PDMS sensor(s) near the front of the bite surface where the incisors are positioned in the mouth, left and right sides of the bite surface where the bicuspid or the pre-molars are positioned in the mouth, and in the left and right back portions of the bite surface where the molars are positioned in the mouth. This array arrangement may be merely exemplary and is not meant to be limiting. The array may be formed in a variety of orientations to capture the various bite forces and shear forces.

At step 708, the 3D printed dentition may be formed by taking a mold of a patient's dentition and generating a 3D dentition model from the mold of the patient's teeth. The 3D printed dentition may include an upper dentition portion and a lower dentition portion.

Once the sensor bed may be created and the 3D printed dentition is formed, the sensor device is calibrated at step 710. At step 710 of method 700, the dental appliance comprising the layered sensor bed that may contain the silver (Ag) impregnated PDMS sensor(s) arranged in an array along the bite surface may be inserted in between an upper dentition portion and a lower dentition portion of a 3D printed dentition formed at step 708. The dental appliance may further comprise a bite surface, a wire harness, and an alignment guide. The dental appliance and 3D printed dentition may be placed within the calibration arbor press for calibration.

The bite surface of the dental appliance may include an upper bite surface portion that makes contact with the upper dentition portion and a lower bite surface portion that makes contact with the lower dentition portion of the 3D printed dentition. Once the 3D printed dentition may be placed on the calibration arbor press and the dental appliance is inserted such that contact is made between the bite surface and the upper dentition portion and lower dentition portion of the 3D printed dentition, the silver (Ag) impregnated PDMS sensor(s) may be calibrated to typical forces.

The calibration arbor press may contain a torque limited calibration handle which operates as a torque-wrench style press. The torque limited calibration handle may be lowered pressing the upper dentition portion and lower dentition portion of the 3D printed dentition onto the upper bite surface portion and lower bite surface portion, respectively, of the dental appliance. By pressing the upper dentition portion and lower dentition portion of the 3D printed dentition onto the upper bite surface portion and lower bite surface portion of the dental appliance, the 3D printed dentition may produce a typical scenario for the silver (Ag) impregnated PDMS sensor(s) arranged in an array to detect the bite force between the upper dentition portion and lower dentition portion of the 3D printed dentition.

At step 712 of method 700, the force vector field may be calculated. The silver (Ag) impregnated PDMS sensor(s) may detect the bite force at specific points of the upper dentition portion and lower dentition portion of the 3D printed dentition by detecting the force on the sensors on various points of the silver (Ag) impregnated PDMS sensor(s) array. Each of the silver (Ag) impregnated PDMS sensor(s) may produce a change in electrical voltage which is used to quantify the strain on the upper dentition portion and lower dentition portion. The change in electrical voltage may be transmitted via the electrodes through the wire harness to a computing device. The computing device may include a controller, which further contains a processor. The controller may receive the change in electrical voltage from the electrodes via the wire harness. The processor may use the change in electrical voltage to calculate the strain and produce the strain calculation value. The force on each individual silver (Ag) impregnated PDMS sensor(s) in the array may be estimated from the strain calculation value and then added to the size of the silver (Ag) impregnated PDMS sensor(s). In some instances, the force calculated for each silver (Ag) impregnated PDMS sensor(s) may provide a more accurate determination of the direct force on each tooth, and/or vector of travel of any particular point on the tooth as it moves functionally against the opposing tooths surface. The vector of travel, for example, may be measured in the z-axis direction, pinpointed at various points of the patient's dentition. In other instances, the combination of forces calculated temporally for each silver (Ag) impregnated PDMS sensor(s) may provide for an accurate determination of the trajectory of the angle of the teeth coming together during a bite, and/or vector of travel of any particular point on the tooth as it moves functionally against the opposing tooths surface, such as in the x or y-axis direction(s), combined with the measurements of force in the z-axis direction, at various points of the patient's dentition. In certain instances, measuring the trajectory of the teeth coming together as a patient bites down or vector of travel of any particular point on the tooth as it moves functionally against the opposing tooths surface may allow for the calculation of the screw axis of the jaw. The screw axis, for example, may be on the axis of a four-bar-linkage design or linkage design. The strain calculation value may be added to the size of the silver (Ag) impregnated PDMS sensor(s) to generate the output value.

Step 714 of method 700 displays the results to a user. The output value may be transmitted from the controller to a display for a user. The display may be, but is not limited to, an interactive screen, a touchscreen, a graphical user interface, or the like. The user may interpret the output value to determine the force of a patient's bite at various pinpointed locations of the patient's dentition based on the bite force calculated for each silver (Ag) impregnated PDMS sensor(s). The force calculated for each silver (Ag) impregnated PDMS sensor(s) may provide a more accurate determination, pinpointed at various points of the patient's dentition.

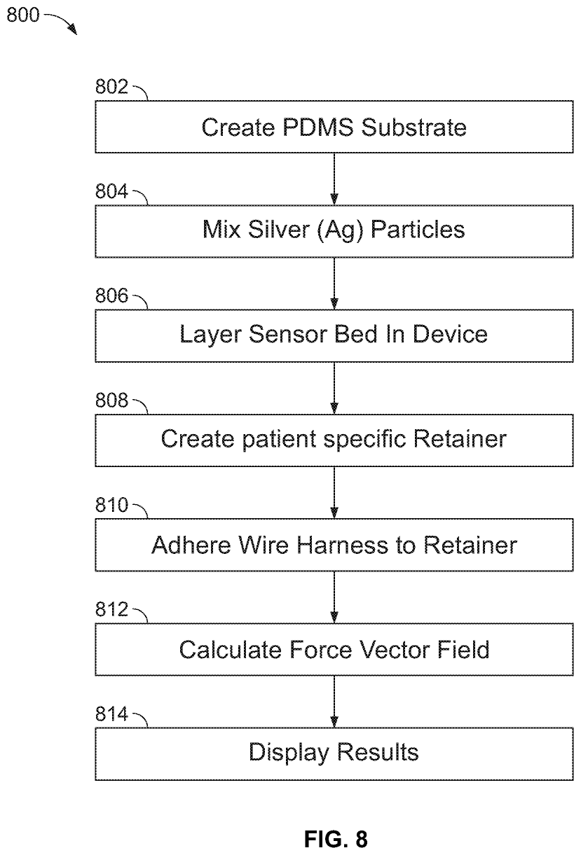

FIG. 8 illustrates a block diagram of a method 800 of estimating the force on the silver (Ag) impregnated PDMS sensor(s) to determine aspects of a patient's bite.

In method 800, at step 802, the sensor substrate may be created. In an instance, the sensor substrate may be a liquid PDMS substrate. In an instance, the sensor substrate may be highly flexible and moldable. The sensor substrate may be impregnated with electrically conductive materials. The sensor substrate may have the ability to be cut after formation. The sensor substrate may even be easily adhered to itself. The sensor substrate may have electrodes placed during the curing phase. The electrodes receive electrical activity which may be electrical signals and transmit the electrical signals to a controller, which may be located locally or on a remote computing device. The sensor substrate may be reusable, easy to clean and/or maintain clean.

At step 804, the sensor substrate may be mixed with a conductive impregnated material forming a silver (Ag) impregnated PDMS layer. The sensor substrate may be mixed uniformly with silver (Ag) particles forming silver (Ag) impregnated PDMS sensor(s). In some instances, the silver (AG) particles 126 may be <250 μm. In another instance, for example, the silver (AG) particles 126 may be between 2-3.5 μm or between 5-8 μm. In some instances, the process of uniformly mixing the silver (Ag) particles with the sensor substrate may include bulk silver (Ag) powder, which may be preliminarily refined by grinding, adding ethanol, and gently stirring the mixture. In some instances, the silver (Ag) particles may be completely dispersed in the liquid mixture by ultrasonication for 30 minutes. In other instances, the silver (Ag) particles 126 may be completely dispersed in the liquid mixture by ultrasonication for a longer or shorter length of time. The ethanol solution on top may be removed with a pipette until the silver (Ag) particles settled at the bottom of the container. When the ethanol is completely volatized, a PDMS prepolymer which may be added to the container. In some instances, the PDMS prepolymer may be a Sylgard 160. In another instance, the PDMS prepolymer may be a harder or softer material. In some instances, the composite may be mixed in a planetary mixer. In some instances, a few drops of a PDMS curing agent may be used between the prepolymer and curing agent. In other instances, more or less PDMS curing agent may be used. The amount and type of PDMS curing agent required may vary according to the manufacturer's instructions. The mixture with the PDMS curing agent may be further mixed again using the planetary mixer. The liquid composite may be poured onto a silicon wafer and spin-coated to form a thin film where the thickness of the film depends on the spin-coating speed and time. In some instances, the thickness may be within a target thickness range of 2-5 mm. In another instance, the thickness may be greater or less than the target thickness. The uncured silver (Ag) impregnated PDMS film may be baked as part of the curing process before being peeled off from the edge of the silicon wafer, thus, forming the silver (Ag) impregnated PDMS sensor(s), which may be easily stretched or twisted and will return to its original state when the tension is fully released.

The uniform mixing of silver (Ag) particles in the sensor substrate may produce a strain gauge effect when PDMS is subjected to forces. In one instance, for example, the conductive impregnated material may have a strain gauge factor of as high as 939 when stretched to 36%. In other instances, the strain gauge factor may be greater or less. In yet other instances, the strain gauge factor may vary based on the location of the device. In one instance, for example, the strain gauge factor may be lower near the molar section of the device to measure greater force in the molar region. The silver (Ag) impregnated PDMS sensor(s) may detect force as low as 0.1 N with a minimum resolution for detection is less than 0.02 N. The strain gauge factor may be the ratio of change in electrical resistance to change in length. The strain gauge factor may be tested and controlled by a high-precision force tester. The strain gauge factor may be used to characterize the sensitivity of the stretchable silver (Ag) impregnated PDMS sensor(s). The results of the resistance change rate and strain with various filling ration and thickness may be characterized in a resistance change rate of

$$\frac{Δ R}{{R}_{0}}$$

versus strain

$$\frac{Δ L}{{L}_{0}} .$$

In general, the resistance change rate may increase at each measurement value. In one instance, for example, the resistance change rate and strain with various filling ration may be measured at 150 wt. %, 175 wt. %, and 200 wt. %, while the thickness may be measured at 400 rpm, 600 rpm, and 800 rpm. In this instance, a film has a 175 wt. %-600 rpm may display different sensing characteristics, which may be divided into three different regions. In this instance, for example, Region 1: Gauge Factor (GF) GF₁=10 (ε=0-24%), Region 2: GF₂=71 (ε=24-32%), and Region 3: GF₃=550 (ε=32-36%). In this exemplary instance, Region 1, the resistance may be relatively stable. However, in this exemplary instance, the resistance change rate may drastically increase with each subsequent region. In other instances, the resistance change rate may vary only slightly. In another instance, the film has a 200 wt. %-800 rpm may display different sensing characteristics, which may similarly be divided into three different regions. In another instance, for example, Region 1: Gauge Factor (GF) GF₁=9 (ε=0-32%), Region 2: GF₂=50 (ε=32-40%), and Region 3: GF₃=157 (ε=40-48%). In yet other instances, the gauge factor may produce different results depending on the region.

The silver (Ag) impregnated PDMS layer may produce a field of strains which are converted to forces by a controller on a computing device using a software. In an instance, the data processing unit may be configured to convert the strain to stress using modulus of elasticity of the PDMS. The modulus may be a design factor similar to the hardness of the material. The data processing unit may be configured to convert stress to force by multiplying by the surface area of the sensor. The plurality of silver (Ag) impregnated PDMS sensor(s) may be arranged in an array. The array may be in a variety of orientations to capture various bite forces and shear forces.

Further in another instance, the electrodes may be arranged in an array in a variety of orientations to capture electrical voltage changes to quantify the strain on the silver (Ag) impregnated PDMS sensor(s). The electrodes may be connected via wire harness leads. The wire harness leads are further connected to the wire harness 506. The electrodes may receive a signal(s) from the silver (Ag) impregnated PDMS sensor(s) and transmit the signal(s) via the wire harness leads to the wire harness 506. The wire harness 506 may be connected to a controller with a processor for processing the signal(s). The controller automatically may receive the signal(s) via the wire harness 506. The processor of the controller may automatically process the received signal(s), which represent a change in electrical voltage and processes the change in electrical voltage in order to calculate the strain and produce the strain calculation value. The processor may estimate the force on each individual silver (Ag) impregnated PDMS sensor(s) in the array using the strain calculation value and then adding the size of the silver (Ag) impregnated PDMS sensor(s). The strain calculation value may be added to the size of the silver (Ag) impregnated PDMS sensor(s) to generate an output value. The output value may be transmitted from the controller to a display for a user. The display may be, but is not limited to, an interactive screen, a touchscreen, a graphical user interface, or the like. The user may interpret the output value and/or vector of travel of any particular point on the tooth as it moves functionally against the opposing tooths surface to determine the force of a patient's bite. The output value and/or vector of travel is interpreted by a user at various pinpointed locations of the patient's dentition based on the bite force calculated for each silver (Ag) impregnated PDMS sensor(s). The output value may be used to assess the health or function of the dentition. In one instance, for example, a user may analyze changes in bite force due to therapeutic treatment of temporomandibular disorders, e.g., deprogramming methods or devices used in dentistry, which could yield changes in bite force values. In another instance, a comprehensive bite analysis may be produced to evaluate and analyze the patient's dentition for various issues. The force calculated for each silver (Ag) impregnated PDMS sensor(s) may provide a more accurate determination, pinpointed at various points of the patient's dentition.

At step 806, the sensor bed of the device may be created by layering the silver (Ag) impregnated PDMS sensor(s) with a non-conductive (inert) PDMS layers. The silver (Ag) impregnated PDMS layer may produce a field of strains which are converted to forces by a controller on a computing device using a software. In an instance, the data processing unit may be configured to convert the strain to stress using modulus of elasticity of the PDMS. The modulus may be a design factor similar to the hardness of the material. The data processing unit may be configured to convert stress to force by multiplying by the surface area of the sensor. The plurality of silver (Ag) impregnated PDMS sensor(s) may be arranged in an array along the bite surface, thereby having silver (Ag) impregnated PDMS sensor(s) near the left and right sides of the bite surface where the bicuspid or the pre-molars are positioned in the mouth, and/or in the left and right back portions of the bite surface where the molars are positioned in the mouth. This array arrangement may be merely exemplary and is not meant to be limiting. The array may be formed in a variety of orientations to capture the various bite forces and shear forces.

At step 808, the patient specific retainer may be formed by taking a mold of a patient's teeth and generating a patient specific retainer from the mold of the patient's teeth. The patient specific retainer has an upper dentition portion and a lower dentition portion. In an instance, the patient specific retainer may be, but is not limited to, a clear custom-made retainer, such as Invisalign.

Once the sensor bed of the device is created and the patient specific retainer is formed, the sensor device may be adhered to the patient specific retainer at step 810. At step 810 of method 800, the device comprising the layered sensor bed with the silver (Ag) impregnated PDMS sensor(s) arranged in an array along the bite surface may be mounted to the patient specific retainer. The device may be mounted in between an upper dentition portion and a lower dentition portion of the patient specific retainer formed at step 808.

The device may further comprise a bite surface, a wire harness, and an alignment guide. The bite surface of the device has an upper bite surface portion in contact with the upper dentition portion of the patient specific retainer and a lower bite surface portion in contact with the lower dentition portion of the patient specific retainer. Once the patient specific retainer with the device adhered may be placed within the mouth of the patient such that contact is made between the bite surface and the upper dentition portion and lower dentition portion of the patient specific retainer, the patient may be permitted to bite down on the patient specific retainer. When the patient bites with the device adhered to the patient specific retainer, the patient's bite may produce a typical scenario for the silver (Ag) impregnated PDMS sensor(s) arranged in an array to detect the bite force between the upper dentition portion and lower dentition portion of the patient specific retainer.

At step 812 of method 800, the force vector field may be calculated. The silver (Ag) impregnated PDMS sensor(s) may detect the bite force at specific points of the upper dentition portion and lower dentition portion of the patient specific retainer by detecting the force on the sensors on various points of the silver (Ag) impregnated PDMS sensor(s) in the array. Each of the silver (Ag) impregnated PDMS sensor(s) may produce a change in electrical voltage which is used to quantify the strain at various points of the upper dentition portion and lower dentition portion of the patient specific retainer. The change in electrical voltage may be transmitted via the electrodes through the wire harness to a computing device. The computing device may include a controller, which further contains a processor. The controller may receive the change in electrical voltage from the electrodes via the wire harness. The processor may use the change in electrical voltage to calculate the strain and produce the strain calculation value. The force on each individual silver (Ag) impregnated PDMS sensor(s) in the array can then be estimated from the strain calculation value and then added to the size of the silver (Ag) impregnated PDMS sensor(s). In some instances, the force calculated for each silver (Ag) impregnated PDMS sensor(s) may provide a more accurate determination of the direct force on each tooth, and/or vector of travel of any particular point on the tooth as it moves functionally against the opposing tooths surface. The vector of travel, for example, may be measured in the z-axis direction, pinpointed at various points of the patient's dentition. In other instances, the combination of force calculated temporally for each silver (Ag) impregnated PDMS sensor(s) may provide for an accurate determination of the trajectory of the angle of the teeth coming together during a bite, and/or vector of travel of any particular point on the tooth as it moves functionally against the opposing tooths surface, such as in the x or y-axis direction(s), combined with the measurements of force in the z-axis direction, at various points of the patient's dentition. In certain instances, measuring the trajectory of the teeth coming together as a patient bites down or vector of travel of any particular point on the tooth as it moves functionally against the opposing tooths surface may allow for the calculation of the screw axis of the jaw. The screw axis, for example, may be on the axis of a four-bar-linkage design or linkage design. The strain calculation value may be added to the size of the silver (Ag) impregnated PDMS sensor(s) to generate the output value.

Step 814 of method 800 displays the results to a user. The output value may be transmitted from the controller to a display for a user. The display may be, but is not limited to, an interactive screen, a touchscreen, a graphical user interface, or the like. The user may interpret the output value to determine the force exerted by a patient's bite at various pinpointed locations of the patient's dentition based on the bite force calculated for each silver (Ag) impregnated PDMS sensor(s). The force calculated for each silver (Ag) impregnated PDMS sensor(s) may provide a more accurate determination, pinpointed at various points of the patient's dentition.

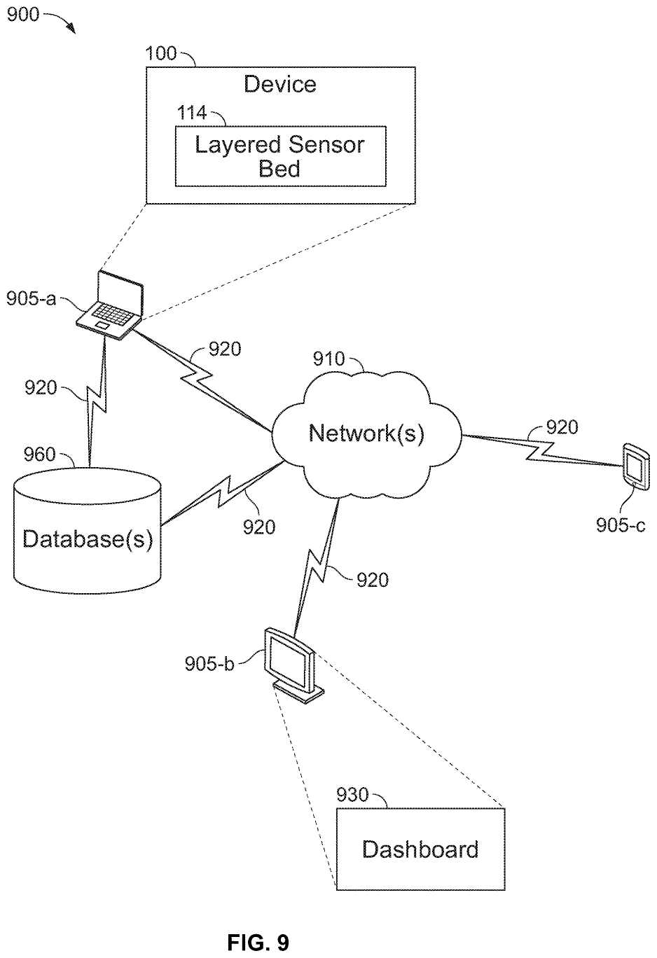

Referring to FIG. 9, which represents a schematic of a computing environment including a device for measuring bite force in accordance with one or more aspects of the present disclosure. The computing environment 900 may include one or more computing devices 905a through 905-c (collectively referred to as computing devices 905), at least one network 910, and at least one database 960. The at least one network 910 may be in communication with one or more computing devices 905 via one or more communication links 920, which may be via other communication networks. The one or more computing devices 905 may be in communication with each other via the network 910 or they may directly communicate via peer-to-peer or side link communications, for example. The computing devices 905 may be, or may include aspects of, the computing device as described in FIGS. 1-8.

The one or more computing devices 905 may be any type of computing devices, such as a desktop computer, laptop, mobile phone, tablet, personal digital assistant (PDA), tablet, personal computer, a workstation, a mainframe computer, a supercomputer, a server, or other electronic device. The one or more computing devices 905 may be enabled with Internet or other network access and may be locatable by the network 910. In one aspect, the one or more computing devices 905 may be configured to transmit instructions to control various devices, receive data or other signals from external devices, and transmit the received data or other data to the network 910, the one or more databases 960, a server, or another remote computing device 905 (e.g., another database) of the computing environment 900.

As illustrated in the example of FIG. 9, the computing device 905-a may be communicatively connected to a device 100. The device 100 may include a layered sensor bed 114. In some instances, the device 100 may be, or may include aspects of, the device 100 as described in FIGS. 1-3. In other instances, the device 100 may be, or may include aspects of, the device 400 as described in FIG. 4. In yet other instances, the device 100 may be, or may include aspects of, the device 500 as described in FIGS. 5-6. Similarly, the layered sensor bed 114 may be, or may include aspects of, the layered sensor bed 114 as described above in FIGS. 2A and 2B. For example, the device 100 may include a dental appliance 102. The dental appliance 102 may include a layered sensor bed 114 and a plurality of electrodes 116, among others components, as described in FIGS. 2A and 2B. The layered sensor bed 114 may comprise a silver (Ag) impregnated PDMS layer 118 and a non-conductive (inert) PDMS layer 120, among other components as described in FIGS. 2A and 2B.

As illustrated in the example of FIG. 9, the computing device 905-b may include a output 930. As described herein, the output 930 may by a program, an application, program interface (API), a graphical user interface (GUI), etc., that may provide interactive output to a user or another computing device 905. For example, the output 930 may be a GUI that is displayed to a user at a display device of the computing device 905-b. The output 930 may present information related to the device 100, including information from the layered sensor bed 114. The output 930 may output notifications or alerts related to the device 100, such as when bite force is detected, when the device 100 has received a bite force reading, or for other reasons. Although the output 930 is shown on the computing device 905-b, in other examples, the output 930 could be on the same computing devices 905 as the device 100.

The one or more computing devices 905 may interface with the one or more databases 960. The one or more databases 960 may be used to store data related to the device 100, such as, information from the PDMS layer 118. The different types of data may be stored in a single database 960 or may be stored in separate databases 960, or any combination. Each of the one or more databases 960 may be any type of centralized database, distributed database, or the like. For example, a centralized database may reside in one central location with may be accessed by carious users, computing devices 905, or applications via a network, such as the at least one network 910. In another aspect, a distributed database may be spread across multiple servers or nodes in the network 910, with each server managing part of the database 960. Within a distributed database, a user may retrieve data seamlessly, similar to if it was stored within a centralized database.

The at least one network 910 may be a local area network (LAN), a wide area network (WAN), or other types of networks, supporting protocols like transmission control protocol/Internet protocol (TCP/IP) over Ethernet. In other examples, the at least one network 910 may include an Automatic Data Processing (ADP) device coupled to a plurality of server devices that may host a plurality of databases and a plurality of client devices via the at least one network 910. The ADP device may store one or more applications that can include executable instructions that, when executed by the ADP device, cause the ADP device to perform desired actions, such as to transmit, receive, or otherwise process network messages, for example, and to perform other actions described and illustrated herein with reference to the figures. The applications may be implemented as modules or components of other applications. Further, the applications can be implemented as operating system extensions, modules, plugins, or the like.

In some instances, the at least one network 910 may be a cloud network. The at least one network 910 may serve as a system hub, providing computational resources and storage capabilities. The at least one network 910 may include resources to manage and deliver computing services over the Internet or other communication form. The network 910 may be executed within or as a virtual machine or a virtual server that may be managed in a cloud-based computing environment. Also, the application(s), and even the ADP device itself, may be located in one or more virtual server(s) running in a cloud-based computing environment rather than being tied to one or more specific physical network computing devices. For example, the application(s) may be running in one or more virtual machines (VMs). In some examples the network 910 may include elements such as data centers, virtualized resources, cloud services, and security tools. In some aspects, the network 910 may utilize data centers that also host servers capable of processing, storing, and managing network traffic, allowing centralized control without on-site hardware.

Additionally, in some examples, elements within the computing environment 900 may include components such as processors, memory, displays, input devices, medium readers, network interfaces, and output devices. The processors may be configured to execute software instructions for performing functions described herein, in addition to other functions such as receiving queries, analyzing the queries, retrieving resolutions, and displaying results.

In other examples, the computing environment 900 of FIG. 9 may include computational and storage capabilities such as processing power, memory, networking, and storage required for the computational success of one or more computing programs. In some instances, the computing environment 900 may integrate cloud computing where applications may access large amounts of computing power on demand, allowing them to scale resources up or down based on computational and storage demands.

According to techniques described herein, in some cases real-time input data quality checking and correction may be facilitated by integrating one or more devices 100, network 910, one or more databases 960, one or more computing devices 905 thereby reducing the need for manual intervention or for signaling between devices. In some examples, the device 100 may assist in data management, software development, or be used as a tool for other types of tasks.

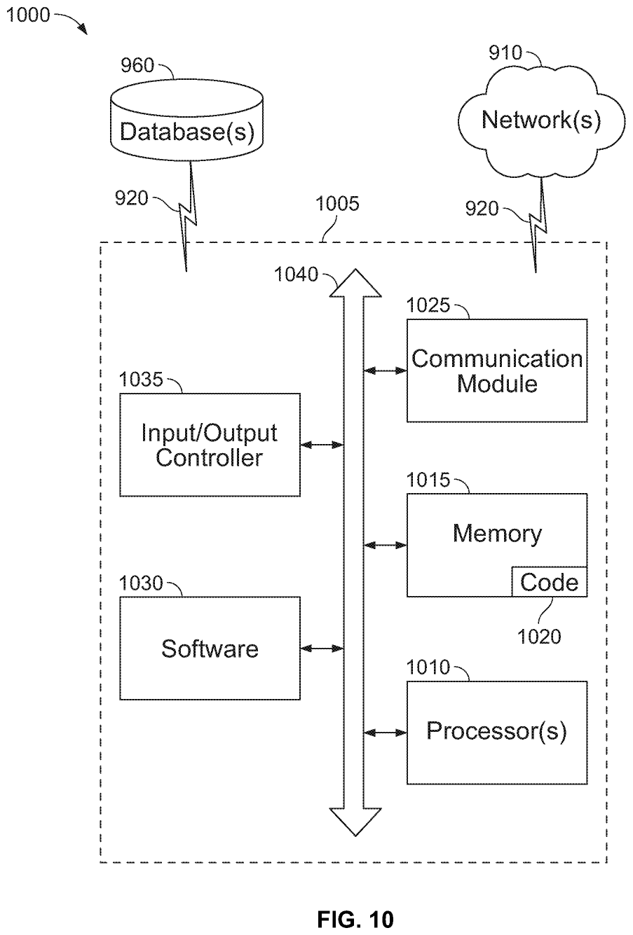

FIG. 10 represents a schematic block diagram of a computer system 1000 for a bite force measurement device 100 according to a disclosed example. The system 1000 may include a computing device 905, at least one network 910, and at least one database 960. The computing device 1005 may be communicatively connected to the network 910 and the database 960 via a communication network 920 or other similar communication link. In some instances, the computing device 1005 may be, or may include aspects of, the one or more computing device 905 as described in FIG. 9. In some instances, the at least one network 910 may be, or include aspects of, the at least one network 910 of FIG. 9. In some instances, the communication network 920 may be, or include aspects of, the communication network 920 or link of FIG. 9.

The computing device 1002 may include at least one processor 1010, one or more memory units 1015 having software code 1020 stored thereon, a communication module 1025, the device 100, an input/output (I/O) controller 1035, and at least one bus 1040. In some instances, one or more devices (including the device 100 in some aspects) may be distributed among one or more computing devices 1005 or stored in a server or database of a network (e.g., such as the at least one database 960 or the at least one network 910). In some instances, one or more bite force measurement devices may be located within the computing device 1005, while additional bite force measurements may be located external to the device 1005 on one or more other devices.

The at least one processor 1010, in some instances, may be a general-purpose processor or may be part of an application-specific integrated circuit (ASIC). In other instances, the at least one processor 1010 may be a microprocessor, a microcomputer, a processor chip, a controller, a microcontroller, a digital signal processor (DSP), a state machine, or a programmable logic device (PLD). Additionally, any processor 1010 described herein may be provided in the form of a single processor, multiple processors, or parallel processors. Multiple processors may be included in or coupled to, single device or multiple devices. In some instances, the processor 1010 may be, or may include aspects of, the processor as described in FIGS. 1-8.

The one or more memory units 1015 may be provided in the form of a single memory unit or multiple memory units and may be one or more articles of manufacture and/or machine components. The one or more memory units 1015 may include static memory, dynamic memory, or both in communication. In some examples, the one or more memory units 1015 may be one or more tangible storage mediums that can store data and executable instructions and may be non-transitory during the time instructions are stored therein. The one or more memory units 1015 may be volatile or non-volatile, secure or encrypted, and unsecure or unencrypted. The software code 1020 stored in the one or more memory units 1015 may include the software code for the device 100 and the other components to operate aspects of the computing environment 1000.

The communication module 1025 may facilitate communication between the device and the network 110 and/or communication between the various components of the computing device 1005 and the network 910 via at least one wired or wireless communication network and/or communication links 920. In some cases, the communication module 1025 may be implemented as part of the at least one processor 1010.

In some instances, the device 100 may be an example of, or may include aspects of, the device 100 FIGS. 1-3. In other instances, the device 100 may be an example of, or may include aspects of, the device 400 FIG. 4. In yet other instances, the device 100 may be an example of, or may include aspects of, the device 500 FIGS. 5-6. In some instances, the device 100 may be a single unit or multiple units. In some aspects, the device 100 may be provided in the form of one or more. In other instances, the device 100 may include separate modules within a single system. The device 100 may perform the measurement of bite force described herein. In some cases, the device 100 may be implemented as part of the at least one processor 1010.

In some examples, the I/O controller 1035 may include a system configured to manage input and output of operations for the computing device 1005, enabling efficient and seamless communication between the communication device 1005 and external systems. The I/O controller 1035 may manage input and output signals for the device 100. In some cases, the I/O controller 1035 may represent a physical connection or port to an external peripheral. In some cases, the I/O controller 235 may utilize an operating system such as iOS®, ANDROID®, MS-DOS®, MS-WINDOWS®, OS/2®, UNIX®, LINUX®, or similar operating system. Additionally, or alternatively, the I/O controller 1035 may represent or interact with a modem, a keyboard, a mouse, a joystick, a trackball, a microphone, a touchpad, a touchscreen, a display, a screen, a monitor, a speaker, or similar devices. In some cases, the I/O controller 1035 may be implemented as part of one or more processors 910. In some cases, a user may interact with the computing device 1005 via the I/O controller 1035 or via hardware components controlled by the I/O controller 1035.

In other instances, the I/O controller 1035 may include an input processing component capable of receiving, validating, and preprocessing incoming data from various sources, ensuring that the data is in a compatible format for use by the device 1005. Within some examples, the I/O controller 1035 may incorporate an output generation component designed to format and deliver the results (e.g., list of predicted entities) or recommendations generated by the device 1005 to downstream applications, interfaces, or systems based on the insights from an output of the device 100. Additionally, the I/O controller 1035 may include a data buffering capability to temporarily store input or output data, ensuring stable and synchronized communication between the computing device 1005 and other connected systems, particularly in real-time or high-throughput environments. The I/O controller 1035 may further include error handling mechanisms to detect, report, and address any issues that arise during input or output operations, such as data inconsistencies, transmission failures, or incompatible formats. As described herein, the I/O controller 1035 may be a program, an application program interface (API), a graphical user interface (GUI), etc., that may provide interactive output to a user or another computing device 1005. For example, the I/O controller 1035 may be a GUI that is displayed to a user at a display device of the computing device 905-b. The I/O controller 1035 may present information related to the device 100, including information related to the identification and measurement of bite force. The I/O controller 1035 may output notifications or alerts related to the device 100, such as when an error occurs when identifying and recording a bite force measurement, or when the device 100 has generated a bite force measurement.

The components of the device 1005 may be interconnected and communicate via the at least one bus 1040 or other communication link. The at least one bus 1040 may enable communication via any standard or specification such as peripheral component interconnect, peripheral component interconnect express, parallel advanced technology attachment, or serial advanced technology attachment. The at least one bus 1040 may be provided in the form of a single bus or multiple buses that are operatively interconnected.

In an example, the computing device 1005 may also include or be in communication with and/or operatively connected to at least one database 160 configured to store data. In some examples, the computing device 1005 may be in communication with the database 960 via the at least one wired or wireless communication link 920. The at least one database 960 may also store parameters associated with the input speech data, including a context of the input speech data, implicit and/or explicit attributes associated with the input speech data, metadata from the input speech data, one or more queries related to the input speech data, etc.

In other instances, other configurations are possible. For example, those of skill in the art will recognize, according to the principles and concepts disclosed herein, that various combinations, sub-combinations, and substitutions of the components discussed above can provide appropriate control for a variety of different configurations of bite force devices for a variety of applications.

It should be understood that the implementation of other variations and modifications of this disclosure and its different aspects are apparent to one skilled in the art, and that this disclosure is not limited by the specific instances described. Features described in one instance can be implemented in other instances. The subject disclosure is understood to encompass the present disclosure and any and all modifications, variations, or equivalents that fall within the spirit and scope of the basic underlying principles disclosed and claimed herein.

1. A device that measures a bite force, the device comprising:
a dental appliance comprising a bite surface;
a sensor array disposed on the bite surface, the sensor array comprising a size and a flexible elastomeric sensor substrate containing at least one electrode and a conductive material distributed within the elastomeric sensor substrate, wherein the conductive material produces a strain gauge effect when the sensor array is subject to a compressive force;
a sensor bed layered with at least one layer of the elastomeric sensor substrate containing the at least one electrode and the conductive material, and at least one non-conductive layer;
a sensor calibration device comprising a torque-wrench style press, a 3D printed upper dentition, and a 3D printed lower dentition configured to apply a calibration force to the sensor array; and
a processor configured to calculate a force vector based on a strain calculation derived from the conductive material and the size of the sensor array.

2. The device of claim 1, wherein the elastomeric sensor substrate comprises polydimethylsiloxane (PDMS).

3. The device of claim 1, wherein the elastomeric sensor substrate comprises one or more of: polydimethylsiloxane (PDMS), polyurethane elastomer, styrene-ethylene-butylene-styrene (SEBS), silicone, or a hydrogel.

4. The device of claim 1, wherein the conductive material comprises one or more of: silver particles, silver nanowires, gold nanoparticles, carbon nanotubes, graphene, carbon black, copper nanoparticles, a liquid metal alloy, or a conductive polymer.

5. The device of claim 4, wherein the conductive material comprises silver particles having a particle size of less than 250 μm.

6. The device of claim 5, wherein the silver particles have a particle size between 2 μm and 3.5 μm, or between 5 μm and 8 μm.

7. The device of claim 1, wherein the at least one electrode receives electrical signals based on a change in electrical resistance in the conductive material caused by a compressive force applied to the sensor array.

8. The device of claim 1, wherein the at least one electrode comprises one or more of: gold, platinum, silver, silver-chloride, or carbon.

9. The device of claim 1, wherein the dental appliance further comprises an alignment guide configured to align the dental appliance with a patient's dentition.

10. The device of claim 1, further comprising a wireless communication module configured to transmit electrical signals from the at least one electrode to a remote computing device via one or more of Bluetooth, Wi-Fi, radio frequency identification (RFID), near-field communication (NFC), or radio waves.

11. A device that measures a bite force, the device comprising:
a sensor array comprising a flexible elastomeric sensor substrate containing at least one electrode and a conductive material distributed within the elastomeric sensor substrate, wherein the conductive material produces a strain gauge effect when the sensor array is subject to a compressive force;
a sensor bed layered with at least one layer of the elastomeric sensor substrate containing the at least one electrode and the conductive material, and at least one non-conductive layer;
a wire harness mechanically coupled to the sensor bed and configured to be adhered to a patient-specific dental retainer; and
a processor configured to calculate a force vector based on a strain calculation derived from the conductive material and a size of the sensor array.

12. The device of claim 11, wherein the patient-specific dental retainer is a clear custom-made dental retainer.

13. The device of claim 11, wherein the patient-specific dental retainer comprises a thermoplastic polyurethane (TPU) retainer, a polyethylene terephthalate glycol (PETG) retainer, or a polymethylmethacrylate (PMMA) retainer.

14. The device of claim 11, wherein the elastomeric sensor substrate comprises polydimethylsiloxane (PDMS).

15. The device of claim 11, wherein the conductive material comprises one or more of: silver particles, silver nanowires, gold nanoparticles, carbon nanotubes, graphene, carbon black, copper nanoparticles, a liquid metal alloy, or a conductive polymer.

16. The device of claim 11, wherein the at least one electrode receives electrical signals based on a change in electrical resistance in the conductive material caused by a compressive force applied by an upper dentition of a patient and a lower dentition of the patient on the sensor array.

17. The device of claim 11, further comprising a wireless communication module replacing the wire harness, the wireless communication module configured to transmit electrical signals from the at least one electrode to a remote computing device via one or more of Bluetooth, Wi-Fi, radio frequency identification (RFID), near-field communication (NFC), or radio waves.

18. A method of measuring bite force using a calibration dental appliance device, the method comprising:
providing a dental appliance comprising a bite surface;
creating a sensor array comprising a flexible elastomeric sensor substrate having at least one electrode embedded therein and a conductive material distributed within the elastomeric sensor substrate;
layering a sensor bed with at least one layer of the elastomeric sensor substrate and at least one non-conductive layer;
disposing the sensor bed on the bite surface of the dental appliance;
calibrating the sensor array by applying a calibration force using a sensor calibration device comprising a torque-wrench style press, a 3D printed upper dentition, and a 3D printed lower dentition;
receiving, from the at least one electrode, electrical signals indicative of a change in electrical resistance in the conductive material resulting from a compressive force applied by a patient's bite; and
calculating, by a processor, a force vector based on a strain calculation derived from the electrical signals and a size of the sensor array.

19. The method of claim 18, wherein the elastomeric sensor substrate comprises polydimethylsiloxane (PDMS).

20. The method of claim 18, wherein the conductive material comprises one or more of: silver (Ag) particles, silver nanowires, gold nanoparticles, carbon nanotubes, graphene, carbon black, copper nanoparticles, a liquid metal alloy, or a conductive polymer.
